(19) United States
(12) Patent Application Publication
(10) Pub. No.: US 2009/0069470 A1
(43) Pub. Date: Mar. 12, 2009

(54) Process for the synthesis of n-alkoxyamines

(76) Inventors: Markus Frey, Rheinfelden (CH); Valerie Rast, Basel (CH); Francisco Martinez, Parma (IT); Davide Alvisi, Bondeno (IT)

Correspondence Address: JoAnn Villamizar;Ciba Corporation/Patent Department, 540 White Plains Road, P.O. Box 2005, Tarrytown, NY, 10591, US

(21) Appl. No.: 11/665,885

(22) PCT Filed: Oct. 24, 2005
(86) PCT No.: PCT/EP05/55472

§ 371 Date: Apr. 19, 2007

(30) Foreign Application Priority Data
Nov. 2, 2004 (EP) 04105456.0

(51) Int. Cl. C08K 5/3492; C07D 403/14

(52) U.S. Cl. 524/100; 544/198

(57) ABSTRACT

The present invention relates to novel processes for the preparation of a sterically hindered amine ethers by the transformation of a corresponding oxo-piperidin to a hydroxy or amino substituted sterically hindered amine ether and the preparation of a N-propoxy or N-propenoxy substituted sterically hindered amine and some novel compounds obtainable by these processes. The compounds made by these processes are particularly effective in the stabilization of polymer compositions against harmful effects of light, oxygen and/or heat and as flame-retardants for polymers.

The present invention relates to novel processes for the preparation of a sterically hindered amine ether by the transformation of a corresponding oxo-piperidin to a hydroxy or amino substituted sterically hindered amine ether and the preparation of a N-propoxy or N-propenoxy substituted sterically hindered amine and some novel compounds obtainable by these processes. The compounds made by these processes are particularly effective in the stabilization of polymer compositions against harmful effects of light, oxygen and/or heat and as flame-retardants for polymers.

WO 01/92228 describes a process for the preparation of amine ethers, e.g. N-hydrocarbyl-oxy substituted hindered amine compounds, by the reaction of the corresponding N-oxyl intermediate with a hydrocarbon in the presence of an organic hydroperoxide and a copper catalyst.

WO 03/045919 describes a process for the preparation of amine ethers, e.g. N-hydrocarbyl-oxy substituted hindered amine compounds, by the reaction of the corresponding N-oxyl intermediate with a hydrocarbon in the presence of an organic hydroperoxide and an iodide catalyst.

DE19907945A describes the formation of 1-allyloxy substituted sterically hindered amines from 1-allyl substituted sterically hindered amines by oxidation.

WO 98/54174 and U.S. Pat. No. 5,844,026 describe the reductive amination of a N-cyclohexyloxy-2,2,6,6-tetramethyl-4-oxo-piperidine to the corresponding amine.

A problem of the state of the art processes is that undesirable side products are obtained that are hard to remove from the desired products as amine oxides do not react selectively with saturated hydrocarbons. The processes of the present invention avoid this problem as hydrocarbons with unsaturated carbon-carbon bonds react more selectively than saturated hydrocarbons, i.e. compounds prepared according to the instant processes may be purer. The transformation product of the process of the present invention may easily be purified by standard methods such as distillation. The hydrogenation of the unsaturated carbon-carbon bond and the reduction or reductive amination of the carbonyl group in one reaction step may save one reaction step and may need less solvents and reagents than the state of the art, i.e. this reaction preformed in two separate reaction steps.

The present invention relates to a process for the preparation of a sterically hindered amine ether of the formula (100)

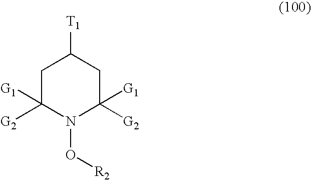

wherein

G₁ and G₂ are independently C₁-C₄alkyl;

R₂ is C₃-C₁₈alkyl or C₅-C₁₂cycloalkyl;

T₁ is hydroxy, —NT₂T₃, —OT₂₂, T₂₀ or a group of formula (102);

T₂ is hydrogen, C₅-C₁₂cycloalkyl or R₄₂; or T₂ is R₄₂ substituted by C₁-C₁₈alkoxy, aryl, hydroxy, carboxy, —CO—O—R₄₂, or —O—CO—R₄₂;

T₃ is hydrogen, C₅-C₁₂cycloalkyl, R₄₂, aryl, -Q-NHT₂ or -Q-NT₂T₂₁; or T₃ is R₄₂ substituted by C₁-C₁₈alkoxy, aryl, hydroxy, carboxy, —CO—O—R₄₂, or —O—CO—R₄₂; or T₃ is aryl substituted by C₁-C₁₈alkoxy, aryl, hydroxy, carboxy, —CO—O—R₄₂, —O—CO—R₄₂ or halogen;

or T₂ and T₃ form together C₄-C₁₁alkylene or C₄-C₁₁alkylene substituted by C₁-C₁₈alkoxy, aryl, hydroxy, carboxy, —CO—O—R₄₂, or —O—CO—R₄₂;

with the proviso that T₂ and T₃ are not benzyl;

R₄₂ is C₁-C₁₈alkyl;

Q is C₂-C₁₈alkylene, C₅-C₁₂cycloalkylene or phenylene;

T₂₂ is —(CO)—(C₁-C₁₆alkylene)_{0 or 1}-(CO)—O-T₂₁;

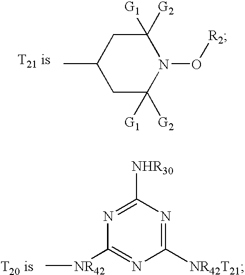

R₃₀ is R₄₂ or R₄₂ substituted by hydroxy; or R₃₀ is —(CH₂)ₙ—NT₂₃-(CH₂)ₚ—NT₂₃-(CH₂)ₙ—NHT₂₃ with one T₂₃ substituent being hydrogen and two T₂₃ substituents being

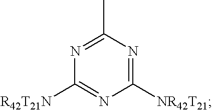

n is 1 to 4;

p is 1 to 3;

the group of formula (102) is

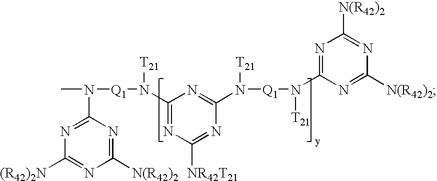

y is 2 to 20;

which comprises transforming a compound of formula (101),

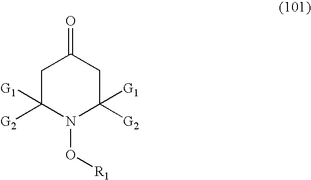

wherein

R₁ is C₃-C₁₈alkenyl or C₅-C₁₂cycloalkenyl,

in one reaction step in the presence of hydrogen and a catalyst into a compound of formula (100) wherein T₁ is hydroxy or —NT₂T₃;

whereby for obtaining compounds with T₁=—NT₂T₃ the transformation is performed in the presence of an amine of formula HNT₂T₃₀;

T₃₀ is hydrogen, C₅-C₁₂cycloalkyl, R₄₂, aryl or -Q-NHT₂; or T₃₀ is R₄₂ substituted by C₁-C₁₈alkoxy, aryl, hydroxy, carboxy, —CO—O—R₄₂, or —O—CO—R₄₂; or T₃₀ is aryl substituted by C₁-C₁₈alkoxy, aryl, hydroxy, carboxy, —CO—O—R₄₂, —O—CO—R₄₂ or halogen;

or T₂ and T₃₀ form together C₄-C₁₁alkylene or C₄-C₁₁alkylene substituted by C₁-C₁₈alkoxy, aryl, hydroxy, carboxy, —CO—O—R₄₂, or —O—CO—R₄₂;

with the proviso that T₃₀ is not benzyl;

and for obtaining a compound of formula (100) with T₁=—OT₂₂, reacting a compound of formula (100) with T₁=hydroxy with an HOOC—(C₁-C₁₆alkylene)_{0 or 1}-COOH or a halide thereof or a methyl ester thereof;

for obtaining a compound of formula (100) with T₁=T₂₀, and R₃₀=R₄₂ or R₄₂ substituted by hydroxy, reacting a compound of formula (100) with T₁=—NT₂T₃, T₂=H, T₃=R₄₂ with a cyanuric halide to yield a compound of formula (103) [step a1], which is subsequently reacted with R₄₂NH₂ or hydroxy-substituted R₄₂NH₂ [step a2];

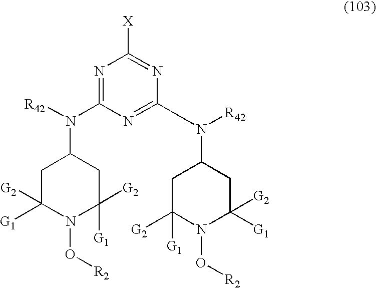

wherein

X is halogen;

for obtaining a compound of formula (100) with T₁=T₂₀ and R₃₀=—(CH₂)ₙ—NT₂₃-(CH₂)ₚ—NT₂₃-(CH₂)ₙ—NHT₂₃,

a compound of formula (103) is reacted with H₂N—(CH₂)ₙ—NH—(CH₂)ₚ—NH—(CH₂)ₙ—NH₂; and

for obtaining a compound of formula (100) with T₁=group of formula (102), reacting a compound of formula (100) with T₁=—NT₂T₃, T₂=H, T₃=R₄₂, with a cyanuric halide to yield a compound of formula (104) [step b1],

which is subsequently reacted with a compound of formula (100) with T₁=—NT₂T₃, T₂=H, T₃=-Q-NHT₂₁, to yield a compound of formula (105) [step b2],

which is subsequently reacted with a compound of formula (100) with T₁=—NT₂T₃, T₂=H, T₃=-Q-NHT₂₁, to yield a compound of formula (106) [step b3],

which is subsequently reacted with a compound 2-X-4,6-bis((R₄₂)₂amino)-s-triazine [step b4].

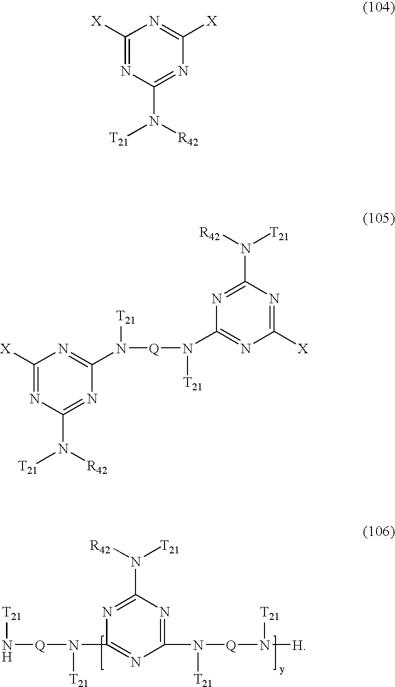

Unless otherwise stated, the reactions described herein are conveniently carried out close to ambient pressure, e.g. between 0.5 and 1.5 bar, especially at about ambient pressure.

The catalyst for the transformation to a compound of formula (100) with T₁=hydroxy or —NT₂T₃ is preferably Rh, Ir, Ru, Pt or Pd on charcoal or Raney-Ni or a reducing agent such as borohydride. 0.0001-0.1 eq., preferably 0.0005-0.01 eq., especially 0.0005-0.005 eq. catalyst is used in this reaction (eq. are given in molar eq. of the compound of formula 101).

The transformation is preferably carried out at a temperature of 35-120° C. and a hydrogen pressure of 6-100 bar, for example at a temperature of 45-110° C. and a hydrogen pressure of 8-60 bar; also of interest is a temperature of 45-110° and a hydrogen pressure of 40-60 bar.

The transformation may be carried out in a solvent, preferably an organic solvent or HNT₂T₃₀, for example HNT₂T₃₀, methanol, ethanol, THF, propanol, i-propanol, butanol, 2-butanol, i-butanol, t-butylmethylether, 1,2-dimethoxyethane, dioxane, di-i-propylether, cyclohexane, hexane or heptane.

A compound of formula (100) with T₁=—OT₂₂ may be obtained by reacting a compound of formula (100) with T₁=hydroxy with an HOOC—(C₁-C₁₆alkylene)_{0 or 1}-COOH or a halide thereof or a methyl ester thereof; such reactions are for example described in C. Ferri, Reaktionen der organischen Synthese, Stuttgart 1978, Georg Thieme Verlag, in particular p. 204 and 447-450 or in R. Larock, comprehensive organic transformations, New York 1989, VCH Verlag, p. 985-987 and the literature cited therein.

The reaction with the halide may be carried out in a neutral, acidic or basic medium, for example in a basic medium such as diluted NaOH, preferably in excess.

The carbonic acid may be reacted in the presence of a catalyst such as inorganic acids, trifluoroacetic acid, arenesulfonacid, ZnCl₂, acidic cation exchanger, SnCl₂ or 2-halogen-1-methylpyridinum salts. The obtained water or diester may be removed from the reaction mixture by distillation. The reaction may be carried out in the absence of a catalyst; in such a case, the reaction may be carried out in the presence of a carbodiimide such as dicyclohexylcarbodiimide.

The reaction with the methyl ester may be carried out in the presence of a catalyst, e.g. NaOAc NaCN, acidic catalyst, (n-C₄H₉)₃SnOR or Ti-, Zr- or Al-alkoxides. Of interest is this reaction being carried out at elevated temperature, for example 50-200° or 50° to the boiling point of the mixture. The compound of formula (100) with T₁=hydroxy may be used in equimolar amount or in excess, for example 2-10, preferably 3-5 molar equivalents. The catalyst may be used in 0.5-0.01 molar eq., preferably 0.25-0.1 molar eq. (eq. are given in molar eq. of the compound of formula (100) with T₁=hydroxy).

A compound of formula (100) with T₁=T₂₀, and R₃₀=R₄₂ or R₄₂ substituted by hydroxy is obtained by reacting a compound of formula (100) with T₁=—NT₂T₃, T₂=H, T₃=R₄₂ with a cyanuric halide to yield a compound of formula (103) [step a1], which is subsequently reacted with R₄₂NH₂ or hydroxy-substituted R₄₂NH₂ [step a2].

Step a1 (as for example described in EP455588; eq. are given as molar eq. of the compound of formula (100) with T₁=—NT₂T₃, T₂=H, T₃=R₄₂):

The cyanuric halide may be a cyanuric chloride (e.g. 0.1-1 eq., especially 0.4-0.6 eq.). The reaction may be carried out in an organic solvent such as xylene, toluene or cyclohexane in the presence of a base such as NaOH, KOH, NaHCO₃ or Na₂CO₃ in for instance 0.5-1.5 eq., especially 0.9-1.1 eq. and optionally a phase-transfer catalyst such as Bu₄NHSO₄ in 0.0001-0.1 eq., for example 0.001-0.01 eq. The reaction temperature may be 60-80° C.

Step a2 (as for instance described in U.S. Pat. No. 5,216,156; eq. are given as molar eq. of the product of step a1):

R₄₂NH₂ or hydroxy-substituted R₄₂NH₂ may be used in 0.5-5 eq. The reaction may be carried out in an organic solvent such as xylene, or in a mixture of xylene and toluene or cyclohexane. Optionally the reaction is carried out in the presence of a base such as NaOH, KOH, NaHCO₃ or Na₂CO₃ in for instance 0.1-1 eq., especially 0.4-0.6 eq. and/or a phase-transfer catalyst such as Bu₄NHSO₄ in 0.0001-0.1 eq, for example 0.001-0.01 eq. The reaction temperature may be 100-130°.

A compound of formula (100) with T₁=T₂₀ and R₃₀=—(CH₂)ₙ—NT₂₃-(CH₂)ₚ—NT₂₃-(CH₂)ₙ—NHT₂₃ may be prepared, as described in EP889085A, by reacting two to four equivalents of a compound of formula (103) with one equivalent of H₂N—(CH₂)ₙ—NH—(CH₂)ₚ—NH—(CH₂)ₙ—NH₂; for example the reaction is carried out in a hydrocarbon solvent with an acid acceptor, such as aqueous sodium hydroxide, to neutralize the hydrochloric acid produced in the reaction.

For example, 2.5 to three equivalents of the compound of formula (103), especially three equivalents of the compound of formula (103), are reacted with one equivalent of H₂N—(CH₂)ₙ—NH—(CH₂)ₚ—NH—(CH₂)ₙ—NH₂. This reaction may be carried out in xylene or in a mixture of xylene and toluene or cyclohexane. A base such as NaOH, KOH, NaHCO₃ or Na₂CO₃ (e.g. 2-4 eq.) and optionally a phase-transfer catalyst (e.g. Bu₄NHSO₄ in for instance 0.02-0.04 eq.) may be used in this reaction (eq. are given as molar eq. of H₂N—(CH₂)ₙ—NH—(CH₂)ₚ—NH—(CH₂)ₙ—NH₂). The reaction temperature may be 100-200° C. The reaction may be carried out at a pressure of 0.5-20 bar, for example 0.5-10 bar, especially 0.5-5 bar, for instance at about ambient pressure.

For obtaining a compound of formula (100) with T₁=group of formula (102), all steps of this reaction may be carried out as for example described in DE19907945.

Step b1 (eq. are given as molar eq. of the compound of formula (100) with T₁=—NT₂T₃, T₂=H, T₃=R₄₂):

A cyanuric halide such as cyanuric chloride may be used in 0.5-1.5 eq., especially 0.9-1.1 eq. Examples for suitable solvents are xylene, toluene or cyclohexane. A base such as NaOH, KOH, NaHCO₃ or Na₂CO₃ in for instance 0.5-1.5 eq., especially 0.9-1.1 eq. and optionally a phase-transfer catalyst such as Bu₄NHSO₄ in 0.001-0.1 eq, for example 0.005-0.05 eq. may be present in this reaction step. The reaction temperature may be 0-40°.

Step b2 (eq. are given as molar eq. of the product of step b1):

The product of step b1, a compound of formula (100) with T₁=—NT₂T₃, T₂=H, T₃=-Q-NHT₂₁ in 0.1-1 eq., especially 0.4-0.6 eq. and a base such as NaOH, KOH, NaHCO₃ or Na₂CO₃ in for instance 0.5-1.5 eq., especially 0.9-1.1 eq. may be reacted at a temperature of 60-80°.

Step b3 (eq. are given as molar eq. of the product of step b2):

The product of step b2 may be reacted with a compound of formula (100) with T₁=—NT₂T₃, T₂=H, T₃=-Q-NHT₂₁ (e.g. 0.5-1.5 eq., especially 0.9-1.1 eq.) and optionally a base such as NaOH, KOH, NaHCO₃ or Na₂CO₃ (for instance 0.5-1.5 eq., especially 0.9-1.1 eq.) at a reaction temperature of for instance 100-200°.

Step b4 (eq. are given as molar eq. of the product of step b3):

The product of step b3 is reacted with 2-X-4,6-bis((R42)2amino-s-triazine (e.g. 0.1-1 eq., especially 0.4-0.6 eq.) optionally in the presence of a base such as NaOH, KOH, NaHCO₃ or Na₂CO₃ (for instance 0.1-1 eq., especially 0.4-0.6 eq.), at a reaction temperature of for example 100-200°.

The steps b3 and b4 may be carried out at a pressure of 0.5-20 bar, for example 0.5-10 bar, especially 0.5-5 bar, for instance at about ambient pressure.

Of interest is a process, wherein

R₂ is C₃-C₁₀alkyl or C₅-C₇cycloalkyl;

T₂ is hydrogen;

T₃ is R₄₂, -Q-NHT₂ or -Q-NT₂T₂₁;

R₄₂ is C₁-C₈alkyl;

Q is C₂-C₈alkylene;

T₂₂ is —(CO)—C₄-C₁₀alkylene-(CO)—O-T₂₁;

n is 2 to 4;

y is 2 to 10

R₁ is C₃-C₁₀alkenyl or C₅-C₇cycloalkenyl and

X is chlorine, bromine or iodine.

For example, X is chlorine.

Of technical interest is R₂ being C₃ or C₈alkyl or C₆cyclohexyl and R₁ being C₃ or C₈alkenyl or C₆cyclohexenyl.

Of interest is R₂ being C₃alkyl, R₁ being C₃alkenyl and T₁ being —NT₂T₃.

An embodiment of the present invention is a process, wherein the compound of formula (101) is obtained by reacting a compound of formula (200) with a C₃-C₁₈alkene or C₅-C₁₂cycloalkene.

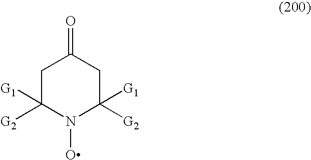

The C₃-C₁₈alkene may be an unbranched alkene, for example a C₃-C₁₈alk-1-ene. Of interest are a C₃-C₁₀alkene or a C₅-C₇alkene, for example C₃ or C₈alkene or C₆cyclohexane, especially C₃alkene.

This process is preferably carried out in the presence of an organic hydroperoxide and optionally a further catalyst.

The further catalyst is preferably selected from the group consisting of scandium, titanium, vanadium, chromium, manganese, iron, cobalt, nickel, copper, zinc, gallium, germanium, yttrium, zirconium, niobium, molybdenum, ruthenium, rhodium, palladium, silver, cadmium, indium, tin, antimony, lanthanum, cerium, hafnium, tantalum, tungsten, rhenium, osmium, iridium, platinum, gold, mercury, thallium, lead, bismuth; the compounds thereof; substituted and unsubstituted ammonium iodides and phosphonium iodides.

The further catalyst may also be quaternary ammonium or phosphonium halogenides such as chlorides or bromides. The structure of the ammonium or phosphonium cation is less important; usually, quaternary ammonium or phosphonium cations contain 4 hydrocarbon residues bonded to the central nitrogen or phosphorus atom, which may be, for example, alkyl, phenylalkyl or phenyl groups. Some readily available materials are tetra-C₁-C₁₂alkylated.

The further catalyst may also be any other iodide compound, including organic and inorganic iodide compounds. Examples are alkaline or alkaline earth metal iodides, or onium iodides such as sulfonium iodides, especially quarternary sulfonium iodides. Suitable metal iodides are, inter alia, those of lithium, sodium, potassium, magnesium or calcium.

The further catalyst is more preferably selected from the group consisting of titanium, vanadium, chromium, manganese, iron, cobalt, nickel, copper, zinc, cerium; the halides and oxides thereof; substituted and unsubstituted ammonium iodides and phosphonium iodides.

The further catalyst is most preferably selected from the group consisting of manganese, iron, cobalt, nickel, copper; the halides thereof; substituted and unsubstituted ammonium iodides and phosphonium iodides, for example substituted and unsubstituted quaternary ammonium or phosphonium iodides, especially tetraalkyl ammonium iodides or tetraphenylphosphonium iodide and triphenylalkylphosphonium iodides.

The further catalyst can be bound to an organic or inorganic polymer backbone, rendering a homogenous or heterogeneous catalytic system.

The further catalysts mentioned above may contain anionic ligands commonly known in complex chemistry of transition metals, such hydride ions (H⁻) or anions derived from inorganic or organic acids, examples being halides, e.g. F⁻, Cl⁻, Br⁻ or I⁻, fluoro complexes of the type BF₄⁻, PF₆⁻, SbF₆⁻ or AsF₆⁻, anions of oxygen acids, alcoholates or acetylides or anions of cyclopentadiene or oxides.

Anions of oxygen acids are, for example, sulfate, phosphate, perchlorate, perbromate, periodate, antimonate, arsenate, nitrate, carbonate, the anion of a C₁-C₈carboxylic acid, such as formate, acetate, propionate, butyrate, benzoate, phenylacetate, mono-, di- or trichloro- or -fluoroacetate, sulfonates, for example methylsulfonate, ethylsulfonate, propylsulfonate, butylsulfonate, trifluoromethylsulfonate (triflate), unsubstituted or C₁-C₄alkyl-, C₁-C₄alkoxy- or halo-, especially fluoro-, chloro- or bromo-substituted phenylsulfonate or benzylsulfonate, for example tosylate, mesylate, brosylate, p-methoxy- or p-ethoxyphenylsulfonate, pentafluorophenylsulfonate or 2,4,6-triisopropylsulfonate, phosphonates, for example methylphosphonate, ethylphosphonate, propylphosphonate, butylphosphonate, phenylphosphonate, p-methylphenylphosphonate or benzylphosphonate, carboxylates derived from a C₁-C₈carboxylic acid, for example formate, acetate, propionate, butyrate, benzoate, phenylacetate, mono-, di- or trichloro- or -fluoroacetate, and also C₁-C₁₂-alcoholates, such as straight chain or branched C₁-C₁₂-alcoholates, e.g. methanolate or ethanolate. Also oxides are possible.

Anionic and neutral ligands may also be present up to the preferred coordination number of the complex cation of the further catalyst, especially four, five or six. Additional negative charges are counterbalanced by cations, especially monovalent cations such as Na⁺, K⁺, NH₄⁺ or (C₁-C₄ alkyl)₄N⁺.

The further catalysts mentioned above may also contain neutral ligands such as inorganic or organic neutral ligands commonly known in complex chemistry of transition metals. Suitable inorganic ligands are selected from the group consisting of aquo (H₂O), amino, nitrogen, carbon monoxide and nitrosyl. Suitable organic ligands are selected from the group consisting of phosphines, e.g. (C₆H₅)₃P, (i-C₃H₇)₃P, (C₅H₉)₃P or (C₆H₁₁)₃P, di-, tri-, tetra- and hydroxyamines, such as ethylenediamine, ethylenediaminotetraacetate (EDTA), N,N-dimethyl-N′,N′-bis(2-dimethylaminoethyl)-ethylenediamine (Me₆TREN), catechol, N,N′-dimethyl-1,2-benzenediamine, 2-(methylamino)phenol, 3-(methylamino)-2-butanol or N,N′-bis(1,1-dimethylethyl)-1,2-ethanediamine, N,N,N′,N″,N″-pentamethyldiethyltriamine (PMDETA), C₁-C₈-glycols or glycerides, e.g. ethylene or propylene glycol or derivatives thereof, e.g. di-, tri- or tetraglyme, and monodentate or bidentate heterocyclic e⁻ donor ligands.

The further catalyst can further contain heterocyclic e⁻ donor ligands which are derived, for example, from unsubstituted or substituted heteroarenes from the group consisting of furan, thiophene, pyrrole, pyridine, bis-pyridine, picolylimine, g-pyran, g-thiopyran, phenanthroline, pyrimidine, bis-pyrimidine, pyrazine, indole, coumarone, thionaphthene, carbazole, dibenzofuran, dibenzothiophene, pyrazole, imidazole, benzimidazole, oxazole, thiazole, bis-thiazole, isoxazole, isothiazole, quinoline, bis-quinoline, isoquinoline, bis-isoquinoline, acridine, chromene, phenazine, phenoxazine, phenothiazine, triazine, thianthrene, purine, bis-imidazole and bis-oxazole.

The sterically hindered aminoxides, also referred to as N-oxyl educts for the instant process which include compounds with at least one group of formula (200), are largely known in the art; they may be prepared by oxidation of the corresponding N—H hindered amine with a suitable oxygen donor, e.g. by the reaction of the corresponding N—H hindered amine with hydrogen peroxide and sodium tungstate as described by E. G. Rozantsev et al., in Synthesis, 1971, 192; or with tert-butyl hydroperoxide and molybdenum (VI) as taught in U.S. Pat. No. 4,691,015, or obtained in analogous manner.

The amount of C₃-C₁₈alkene or C₅-C₁₂cycloalkene is typically a ratio of 1 to 100 moles of C₅-C₁₈alk-1-ene per mole of compound of formula (200) with the preferred ratio being 1 to 50 moles per mole of compound of formula (200), and the most preferred ratio being 1 to 30 moles of C₅-C₁₈alk-1-ene per mole of compound of formula (200).

For example, the amount of organic hydroperoxide is 0.5 to 20 moles per mole of compound of formula (200), with the preferred amount being 0.5 to 5 moles of peroxide per mole of compound of formula (200) and the most preferred amount being 0.5 to 3 moles of peroxide per mole of compound of formula (200).

The organic hydroperoxide used in the process of present invention can be of the formula R—OOH, wherein R usually is a hydrocarbon containing 1-18, preferably 3-18 carbon atoms. R is advantageously aliphatic, for example an alkyl group, preferably C₁-C₁₂alkyl. Most preferably, the organic hydroperoxide is tert-butyl-hydroperoxide or cumyl hydroperoxide.

The preferred amount of further catalyst is from about 0.0001 to 0.5, especially 0.0005 to 0.1 molar equivalent per mole of compound of formula (200), with a ratio of 0.001 to 0.05 moles of further catalyst per mole of compound of formula (200) being the most preferred.

The reaction is preferably run at 0 to 100° C.; more preferably at 20° to 100° C., especially in the range from 20 to 80° C.

The C₅-C₁₈alkene or C₅-C₁₂cycloalkene may serve two functions both as reactant and as solvent for the reaction. The reaction can also be carried out using an inert organic or inorganic solvent.

Such solvent may be used, especially if the further catalyst is not very soluble in the C₅-C₁₈alk-1-ene. Typical inert solvents are acetonitrile, aromatic hydrocarbons like benzene, chlorobenzene, CCl₄, alcohols (e.g. methanol, ethanol, ethylene glycol, ethylene glycol monomethyl ether), or alkanes like hexane, decane etc., or mixtures thereof. Inorganic solvents such as water are possible as well.

The instant process can be run in air or in an inert atmosphere such as nitrogen or argon. The instant process can be run under ambient pressure as well as under reduced or elevated pressure.

There are several variations of the instant process. One variation involves the addition of a solution of organic hydroperoxide to a mixture of the N-oxyl hindered amine, the C₅-C₁₈alkene or C₅-C₁₂cycloalkene and solvent (if used), and optionally further catalyst which has been brought to the desired temperature for reaction. The proper temperature may be maintained by controlling the rate of peroxide addition and/or by using a heating or cooling bath. After the hydroperoxide is added, the reaction mixture is conveniently stirred till the starting amineoxide has disappeared or is no longer being converted to the desired product, e.g. compound of formula (101). The reaction can be monitored by methods known in the art such as UV-VIS spectroscopy, thin layer chromatography, gas chromatography or liquid chromatography. Additional portions of catalyst can be added while the reaction is in progress. After the initial hydroperoxide charge has been added to the reaction mixture, more hydroperoxide can be added dropwise to bring the reaction to completion.

A second variation of the instant process is to simultaneously add separate solutions of the hydroperoxide and the compound of formula (200) to a mixture of the C₅-C₁₈alkene or C₅-C₁₂cycloalkene, solvent (if used) and optionally further catalyst. The compound of formula (200) may be dissolved in water or the solvent used in the reaction, for example an alcohol. Some of the compound of formula (200) may be introduced into the reaction mixture prior to starting the peroxide addition, and all of the compound of formula (200) should be added prior to completing the peroxide addition.

Another variation of the instant process involves the simultaneous addition of separate solutions of the hydroperoxide and of the aqueous or solvent solution of the further catalyst to a mixture of the compound of formula (200), C₅-C₁₈alk-1-ene or C₅-C₁₂cycloalkene, and solvent (if used). Some of the further catalyst may be introduced into the reaction mixture prior to starting the peroxide addition.

Still another variation of the instant process is the simultaneous addition of separate solutions of the hydroperoxide, of the aqueous or solvent solution of the nitroxyl compound, and of an aqueous or solvent solution of the further catalyst to the C₅-C₁₈alk-1-ene or C₅-C₁₂cycloalkene and solvent (if used). A portion of the compound of formula (200) and/or catalyst may be introduced into the reaction mixture prior to starting the hydroperoxide addition. All of the compound of formula (200) should be added prior to completing the hydroperoxide addition.

At the end of the reaction, the residual hydroperoxide may be carefully decomposed prior to the isolation of any products.

Another embodiment of the present invention is a process, wherein the compound of formula (200) is obtained by oxidizing a compound of formula (201).

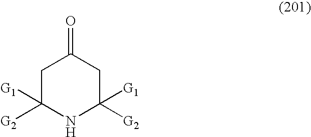

The sterically hindered aminoxides, which include compounds of formula (200), are largely known in the art; they may be prepared by oxidation of the corresponding N—H hindered amine with a suitable oxygen donor, e.g. by the reaction of the corresponding N—H hindered amine with hydrogen peroxide and sodium tungstate as described by E. G. Rozantsev et al., in Synthesis, 1971, 192; or with tert-butyl hydroperoxide and molybdenum (VI) as taught in U.S. Pat. No. 4,691,015, or obtained in analogous manner. Starting compounds of formula (201) are known in the art, are partly commercially available or can be synthesised according to procedures known in the art as for example described in U.S. Pat. No. 4,734,502.

The above-mentioned processes may comprise the conversion of a compound of formula (201) to a compound of formula (100) without the isolation of the intermediate products.

For instance, the above-mentioned processes may comprises the conversion of a compound of formula (200) to a compound of formula (100) without the isolation of the intermediate products.

The compound of formula (101) with R₁ being the group

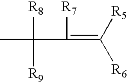

wherein

R₅, R₆, R₇, R₈ and R₉, independently of each other, are H, C₁-C₈alkyl, C₂-C₈alkenyl; and R₇ and R₈ together may also form a chemical bond;

is obtained by a process involving the following: reacting a compound of formula (202) with a compound of formula (203),

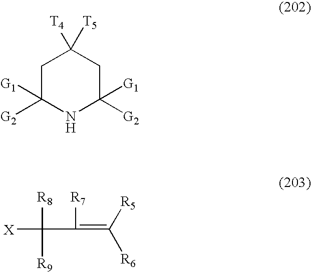

wherein T₄ and T₅ are independently C₁-C₁₈alkoxy; or T₄ is hydroxy and T₅ is hydrogen;

X is halogen;

affording a compound of formula (204);

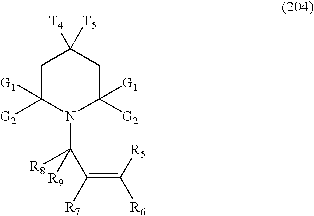

oxidizing the compound of formula (204) in the presence of oxygen, peroxides, permanganates or chlorates affords a compound of formula (205); and

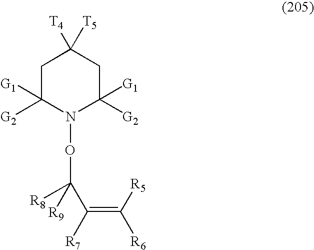

deacetalising the compound of formula (205) with T₄ and T₅ being independently C₁-C₁₈alkoxy or oxidizing the compound of formula (205) with T₄=hydroxy and T₅=hydrogen.

Starting compounds of (202) are known in the art, are partly commercially available or can be synthesised according to procedures known in the art as for example described in EP0748849 A.

The compound of formula (204) may be obtained from the compounds of formulae (202) and (203) as described in C. Ferri, Reaktionen der organischen Synthese, Stuttgart 1978, Georg Thieme Verlag, in particular p. 211-212 and the literature cited therein. The molar ratio of the compound of formula (202) to the compound of formula (203) is 0.5 to 4, preferably 1 to 3, most preferably 1.5 to 2.5. As catalyst, Cu or Pd powder, Cu or Pd salt or phosphine complexes thereof or quarternary ammonium salt such as Bu₄N⁺ salts, for example Bu₄NHSO₄ may be used in catalytic amounts. The reaction may be carried out with or without a solvent. Suitable solvents can be hydrocarbons (e.g. xylene or toluene), alcohols (especially methanol or ethanol), ethers (e.g. tetrahydrofuran) or molar solvents like dimethylformamide or N-methyl-2-pyrrolidone. The reaction temperature may be 20-150°, for example 50-120° or for reactions including a solvent 50° to the boiling point of the solvent. Optionally, a base such as an alkali metal carbonate, hydrogencarbonate or hydroxide, for example Na₂CO₃, NaHCO₃ or NaOH, may be present as a reagent.

X in formula (203) is preferably chlorine, bromine or iodine, most preferably bromine or iodine.

The oxidation to obtain the compound of formula (205) from the compound of formula (204) can be carried out using known oxidants, e.g. oxygen, peroxides or other oxidizing agents such as nitrates, permanganates, chlorates; preferred are peroxides, such as hydrogen peroxide based systems, especially peracids such as perbenzoic acid or peracetic acid. The oxidant is conveniently used in stoichiometric amount or in excess, e.g. using 1-2 moles active oxygen atoms for each compound of formula (204).

The reaction can be carried out in the presence of a suitable solvent, for example an aromatic or aliphatic hydrocarbon, alcohol, ester, amide, ether, or halogenated hydrocarbon; examples are benzene, toluene, xylene, mesitylene, methanol, ethanol, propanol, butanol, dimethylformamide, dimethylsulfoxide, methylene chloride; preferred is a C₁-C₄alcohol, benzene, toluene, xylene, or chlorinated C₁-C₆hydrocarbon.

Temperature and pressure are not critical and depend mainly on the oxidant system used; preferably, temperature is kept during the reaction in the range between −20° C. and +40° C. Conveniently, the pressure is kept close to ambient pressure, e.g. between 0.5 and 1.5 bar; when oxidation is achieved with gaseous oxygen, the pressure of oxygen or oxygen/inert gas may exceed ambient pressure.

Deacetalising the compound of formula (205) with T₄ and T₅ being independently C₁-C₁₈alkoxy may be carried out by known methods as for example described in C. Ferri, Reaktionen der organischen Synthese, Stuttgart 1978, Georg Thieme Verlag, particularly p. 241 or J. March, Advanced organic chemistry, 3. edition, New York 1985, Wiley-Interscience, in particular p. 329-331 or in Th. Greene, protective groups in organic synthesis, John Wiley & Sons Inc., New York 1991, p. 180-183 and the literature cited in these references. The deacetalising may be carried out in an organic solvent as for example tetrahydrofuran in the presence of water and an acid. The acid may be HCl, HBr or HI, especially HCl. Water may be used in excess, i.e. more than one mol water per mol of compound of formula (205). The deacetalising may be carried out with LiBF₄ in wet acetonitrile or in nonaqueous conditions with Me₃SiI in methylenechloride or in chloroform. Of technical interest is the deacetalising using H₂O/HCl. 1-100 eq., preferably 10-50 eq. water, 0.01-10 eq., preferably 0.1-1 eq. HCl and a co-solvent such as THF, MeOH or EtOH is used. The reaction temperature may be 0-80°, preferably 20-50° C.

Oxidizing the compound of formula (205) with T₄=hydroxy and T₅=hydrogen is carried out by known methods such as described in J. March, Advanced Organic Chemistry, John Wiley & Sons, New York, 1992, p. 1167-1171 and the literature cited therein.

Primary oxidants may be, but are not limited to, those being industrially attractive because they are both, cheap and environmentally benign, such as e.g. a catalyst and a further substance selected from the group consisting of oxygen, hydrogenperoxide, a hypochlorite, an alkylhydroperoxide and a carbonyl compound:

a) Oxygen and a catalyst such as a nitroxide (2,2,6,6-tetramethylpiperidine-N-oxide (TEMPO), 4-[C₁-C₁₆alkyl oxy, C₁-C₁₆alkanoyl oxy or aroyl oxy]-TEMPO, Chimassorb® 944 or compound K′ of Example 12), N-hydroxyphtalimide, N,N,N-trihydroxyisocyanuric acid or N-hydroxysaccharin together with one or more of the following co-catalysts: a polyoxometallic acid or its alkali or tetraalkylammonium salt (e.g. H₅[PMo₁₀V₂O₄₀]; tungstates, phosphotungstates, silicotungstates, borotungstates, vanadates, molybdates, phosphomolybdates, silicomolybdates, titanates or silicotitanates); a group VIIA, VIIIA or IB metal, an oxide thereof, a salt thereof (e.g. chlorides, bromides, acetates or acetylacetonates) or a complex thereof (e.g. Pd[PPh₃]₂Cl₂, Pd[PPh₃]₄, Ru[PPh₃]₄H₂, Ru(PPh₃)₃Cl₂ or Cu[1,10-phenantroline]Cl); enzymes such as chloroperoxidase; further co-catalysts or co-additives may be alkali, earthalkali or tetraalkylammonium iodides; symdicarbethoxy hydrazine or diethyl azodicarboxylate; benzoic, 3-chlorobenzoic, phtalic or iso-phtalic acid; alkali hydrogencarbonates or carbonates; hydroquinone or p-benzoquinone; ascorbic acid.

b) Hydrogenperoxide and a catalyst such as a polyoxometallate as described above (e.g. Na₂WO₄) or an enzyme (e.g. chloroperoxidase), together with one or more of the following co-catalysts: a nitroxide as defined above or its deoxygenated precursor (amine); a phase-transfer agent such as tetraalkylammonium halides (especially chlorides, bromides, iodides or hydrogensulfates, e.g. trioctylmethylammonium hydrogensulfate).

c) a hypochlorite and a catalyst such as a nitroxide defined as above together with a co-catalyst such as alkali or earthalkali bromides or iodides or alkali borates.

d) an alkylhydroperoxide (e.g. t-butylhydroperoxide or cumylhydroperoxide) and a catalyst such as Al-, Zr- or Ti-alkoxides (for instance n-propoxides, i-propoxides or t-butoxides, e.g. Zr[OⁿPr]₄, Zr[OⁱPr]₄ or Zr[O^{t}Bu]₄), ZrO(OAc)₂ or an enzyme (e.g. chloroperoxidase).

e) carbonyl compounds such as ketones (e.g. acetone, 2-butanone, 3-pentanone, 4-methyl-2-pentanone, cyclohexanone) and a catalyst such as Al-, Zr- or Ti-alkoxides (e.g. Al[OⁿPr]₃, Al[OⁱPr]₃ or Al[O^{t}Bu]₃, metals (e.g. Pt, Pd, Ru or Raney Nickel) or Ru complexes (e.g. Ru[PPh₃]₄H₂ or Ru(PPh₃)₃Cl₂).

The catalysis may be homogeneous or heterogenous, and the reaction may be homogeneous (one-phase) or heterogeneous (two- or more phases).

In the examples for reactions a) to e), equivalents (eq.) are given as molar equivalents of a compound of formula (205) with T₄=hydroxy and T₅=hydrogen unless otherwise stated.

Examples for oxygen and a catalyst are:

a1) described by R. Neumann et al., J. Org. Chem. 66, 8650-8653 (2001):

0.001-0.1, preferably 0.005-0.05 eq. H₅[PMo₁₀V₂O₄₀]; 0.001-0.1, preferably 0.005-0.05, especially 0.02-0.04 eq. TEMPO; the reaction may be carried out in a solvent such as acetone or a mixture of acetone and 2-butanone, 3-pentanone, 4-methyl-2-pentanone or cyclohexanone; the pressure of oxygen may be 1-10, for example 1.5-5, about 1.5-2.5 atm; the reaction temperature may be 25-125°, preferably 50-110°, especially 90-110°.

a2) described by A. Sheldon et al., J. Am. Chem. Soc. 123, 6826-6833 (2001):

0.001-0.1, preferably 0.005-0.05 eq. RuCl₂(PPh₃)₃; 0.001-0.2, preferably 0.015-0.15 eq. TEMPO; the reaction may be carried out in a solvent such as chlorobenzene; the pressure of oxygen may be 1-20, preferably 5-15, for example 8-12 bar; the reaction temperature may be 25-125°, preferably 50-110°, especially 90-110°.

a3) described by Y. Ishii et al., J. Org. Chem. 65, 6502-6507 (2000):

0.01-0.2, preferably 0.05-0.15 eq. N-hydroxyphtalimide; 0.001-0.1, preferably 0.002-0.05 eq. Co(OAc)₂; 0.01-0.5, preferably 0.02-0.1 eq. m-chlorobenzoic acid; the reaction may be carried out in a solvent such as ethylacetate or in a mixture of ethylacetate and chlorobenzene, acetonitrile or methylacetate; the pressure of oxygen may be 0.5-50, preferably 0.5-25, for example 0.5-2 bar; the reaction temperature may be 0-100°, preferably 20-50°, especially 20-30°.

a4) described by I. Marko et al., J. Org. Chem. 64, 2433-2439 (1999):

0.01-0.5, preferably 0.02-0.1 eq. Cu[1,10-phenantroline]Cl; 0.01-0.5, preferably 0.02-0.1 eq. sym-dicarbethoxy hydrazine or diethyl azodicarboxylate; 0.1-4, preferably 1-3 eq. K₂CO₃; the reaction may be carried out in a solvent such as toluene or a mixture of toluene with chlorobenzene, acetonitrile, ethylacetate or methylacetate; the pressure of oxygen may be 0.5-50, preferably 0.5-25, especially 0.5-1.5 bar; the reaction temperature may be 25-120°, preferably 50-100°, especially 80-100°.

An example of hydrogenperoxide and a catalyst is described by R. Noyori et al., Chem. Commun. 2003, 1977-1986:

0.001-0.1, preferably 0.0015-0.05 eq. Na₂WO₄; 0.001-0.1, preferably 0.0015-0.05 eq. trioctylmethylammonium hydrogensulfate; 1-5, preferably 1-2 eq. H₂O₂ (e.g. aqueous 25-35%); the reaction temperature may be 25-100°, preferably 50-100°, especially 85-95°.

An example of hypochlorite and a catalyst is described by H. van Bekkum et al., Synthesis 10, 1153-1174 (1996):

0.001-0.1, preferably 0.005-0.05 eq. 4-methoxy-TEMPO; 0.01-0.3, preferably 0.05-0.2 eq. KBr; 1-3, preferably 1.1-1.75 eq. NaOCl (e.g. 0.35 molar); the reaction may be carried out in a solvent such as dichloromethane or a mixture of dichloromethane and 1,2-dichloroethane, ethylacetate, methylacetate, chlorobenzene or toluene; the reaction temperature may be −10 to 50°, preferably −5 to 30°, especially −5 to 10°.

An example of an alkylhydroperoxide and a catalyst is described by H. Adam et al., J. Org. Chem. 61, 1467-1472 (1996):

0.01-1, preferably 0.05-0.5 eq. Zr(OⁿPr)₄ or Zr(OtBu)₄; 1-5, preferably 1.5-3 eq. t-BuOOH (e.g. anhydrous); the reaction may be carried out in a solvent such as toluene or a mixture of toluene and cyclohexane, hexane, dichloromethane, chloroform, 1,2-dichloroethane, ethylacetate or methylacetate; optionally the reaction is carried out in the presence of molecular sieves; the reaction temperature may be −25 to 100°, preferably 0-80°, especially 20-50°.

Examples for carbonyl compounds and a catalyst are:

e1) described by J. Bäckvall et al., J. Org. Chem. 61, 6587-6590 (1996):

0.001-0.05, preferably 0.0015-0.03 eq. Ru(PPh₃)₃Cl₂; examples of carbonyl compounds are acetone, 2-butanone, 3-pentanone, 4-methyl-2-pentanone and cyclohexanone, wherein acetone may be used as solvent as well; the reaction temperature may be 25-120°, preferably 50-100°, especially about the reflux temperature of the reaction mixture.

e2) described by Houben-Weyl, Methoden der Organischen Chemie, Georg Thieme Verlag, Stuttgart 1973, vol. 7/2a, p. 714-718 and Org. Synth. IV, 192-195 (1963):

0.01-0.8, preferably 0.05-0.6 eq., especially 0.4-0.6 eq. Al(OtBu)₃ or Al(OiPr)₃; examples of carbonyl compounds are cyclohexanone, acetone, 2-butanone, 3-pentanone and 4-methyl-2-pentanone, usually 1-50, preferably 10-30 eq. of the carbonyl compound is used; a mixture of toluene and chlorobenzene, THF, 1,4-dioxane or 1,2-dichloroethane may be used as solvent; the reaction temperature may be 25-130°, preferably 50-120°, especially about the reflux temperature.

Preference is given to a reaction carried out in presence of alkylhydroperoxides and a catalyst as described above.

Compounds of formula (205) may also be obtained from a compound of formula (202) in analogous manner as the reaction of compound of formula (201) to a compound of formula (200) with consecutive reaction to a compound of formula (101). So compounds of formula (202) may be oxidized and the obtained product may be reacted with a C₃-C₁₈alkene or C₅-C₁₂cycloalkene as described above. Such a reaction sequence is shown in Example 11.

Compounds of formula (205) may be directly converted to compounds of formula (100) by initial imine formation by subsequent hydrogenation. This reaction may be catalyzed by e.g. Sc(OTf)₃ or by La(OTf)₃. Such a reaction is described for example in H. Heaney et al., Synlett. 1998, 640-642.

This invention also relates to a process for the preparation of a compound of formula (300)

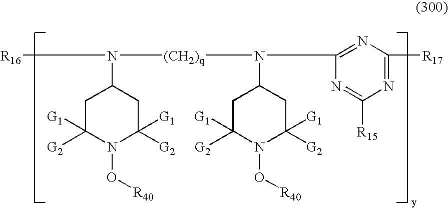

wherein

G₁ and G₂ are independently C₁-C₄alkyl;

R₄₀ is propyl or 2-propenyl;

y is 2 to 20;

q is 2 to 8;

R₁₅ is morpholino, piperidino, 1-piperizinyl, alkylamino of 1 to 8 carbon atoms, —N(C₁-C₈alkyl)T₁₀, or —N(alkyl)₂ of 2 to 16 carbon atoms,

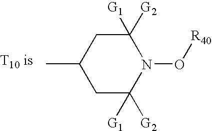

R₁₆ is hydrogen, C₂-C₄acyl, carbamoyl substituted by C₁-C₄alkyl, s-triazinyl substituted once by chlorine and once by R₁₅, or s-triazinyl substituted twice by R₁₅ with the condition that the two R₁₅ substituents may be different;

R₁₇ is chlorine, amino substituted by C₁-C₈alkyl or by T₁₀, —N(C₁-C₈alkyl)T₁₀, —N(alkyl)₂ of 2 to 16 carbon atoms, or the group T₁₃

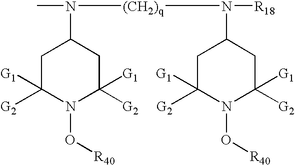

R₁₈ is hydrogen, C₂-C₄acyl, carbamoyl substituted by C₁-C₄alkyl, s-triazinyl substituted twice by —N(alkyl)₂ of 2 to 16 carbon atoms or s-triazinyl substituted twice by —N(C₁-C₈alkyl)T₁₀;

which comprises oxidizing a compound of formula (300) wherein >N—O—R₄₀ is >N—H to a compound of formula (300) wherein —O—R₄₀ is —O, which is subsequently reacted with propene;

and hydrogenating this compound for obtaining a compound of formula (300) with R₄₀=propyl.

Of technical interest are compounds of formula (300) wherein R₁₅ is —N(C₁-C₈alkyl)T₁₀, R₁₆ is s-triazinyl substituted twice by R₁₅=—N(alkyl)₂ of 2 to 16 carbon atoms, R₁₇ is T₁₃, R₁₈ is s-triazinyl substituted twice by —N(alkyl)₂ of 2 to 16 carbon atoms.

Starting compounds of formula (300) wherein >N—O—R₄₀ is >N—H are known in the art, are partly commercially available or can be synthesised according to procedures known in the art as for example described in DE19959619 or CA2191832.

The corresponding amine oxides (compounds of formula (300) wherein —O—R₄₀ is —O) may be obtained as described above for obtaining compounds of formula (200).

Compounds of formula (300) with R₄₀=propenyl may be obtained as described in the process for obtaining compounds of formula (101) from compounds of formula (200) It might be necessary to add some ligands such as 4,4-di-tert-butyl-2,2-dipyridyl to the further catalyst to obtain the desired product.

Advantageously, hydrogenation of compound of formula (300) with R₄₀=propenyl is carried out in the presence of a hydrogenation catalyst.

The hydrogenation catalyst is preferably selected from the group consisting of platinum, palladium, ruthenium, rhodium, Lindlar catalyst, platinum compounds, palladium compounds, ruthenium compounds, rhodium compounds, iridium compounds, nickel compounds, zinc compounds and cobalt compounds.

The hydrogenation catalyst can be bound to an organic or inorganic polymer backbone, rendering a homogenous or heterogeneous catalytic system. Hydrogenation can also be carried out as transfer hydrogenation such as described in S. Murashi et al., Chem. Rev. (1998), 98, 2599-2660 or with further hydrogenation methods such as described in Larock, comprehensive organic transformations.

More preferably, the hydrogenation catalyst is selected from the group consisting of platinum, palladium, ruthenium, platinum compounds, palladium compounds and ruthenium compounds.

Most preferably, the hydrogenation catalyst is selected from the group consisting of platinum, palladium and ruthenium; platinum, palladium and ruthenium immobilized on carbon; PtO₂, Pd—CaCO₃—PbO, RuClH[PPh₃]₃, RhCl[PPh₃]₃ and RuH₂[P(Ph)₃]₄.

The preferred amount of hydrogenation catalyst is 0.0001-0.2 mol per mol of unsaturated amine ether moiety. The hydrogenation reaction is preferably run at 0° to 80° C.; especially in the range 20-60° C. The hydrogen pressure is preferably 1-20 atm, for example 1-5 atm.

In the above-mentioned processes, G₁ and G₂ are for example methyl.

Some of the compounds available by the instant processes are novel and are another embodiment of this invention. These compounds are of formula (400) to (407)

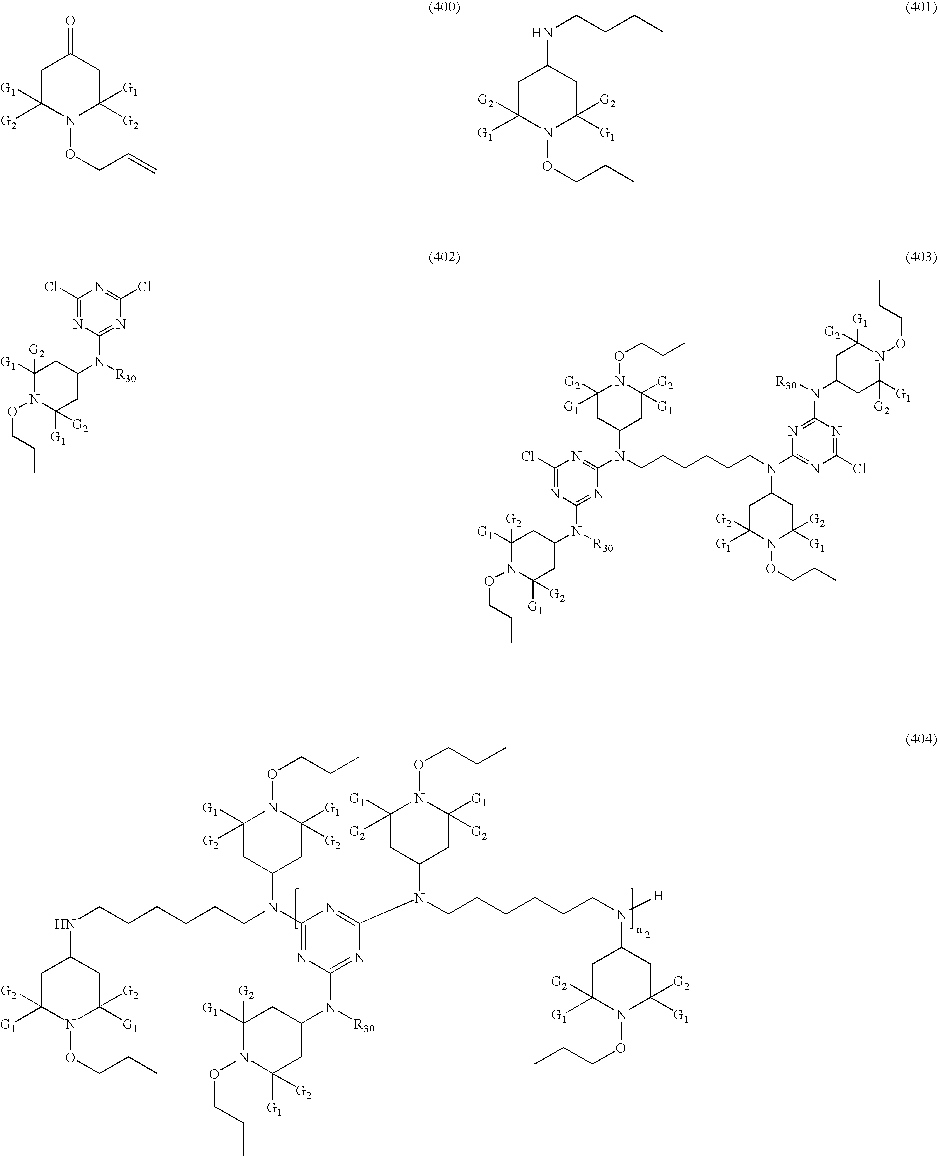

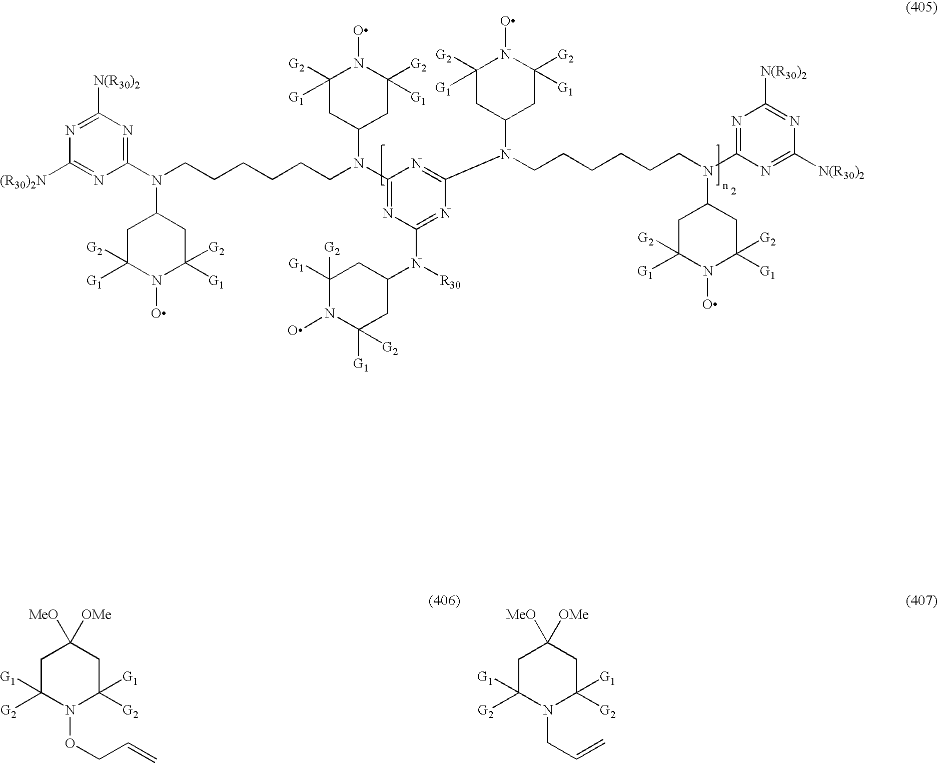

wherein G₁ and G₂ are independently C₁-C₄alkyl;

R₃₀ is C₁-C₈alkyl and

n₂ is 2 to 20.

Of interest is a mixture of compounds of formulae (408) and (409),

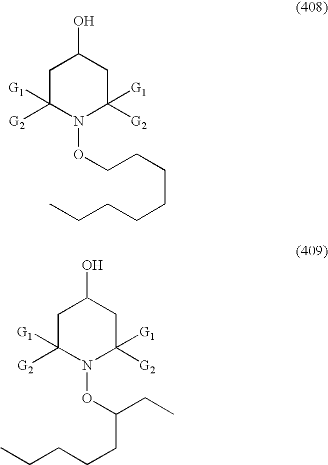

wherein G₁ and G₂ are independently C₁-C₄alkyl.

A mixture of compounds of formulae (408) and (409) is preferred, wherein the ratio of the compound of formula (408) to the compound of formula (409) is from 1:9 to 7:3, in particular from 1:4 to 3:2, for example 3:7 to 1:1, most preferred from 7:13 to 9:11.

Of interest are compounds or a mixture of compounds, wherein G₁ and G₂ are methyl.

Of interest is R₃₀ being butyl.

The instant compounds may be prepared according to one of the processes of this invention.

In the definitions the term alkene comprises, for example propene, and the branched and unbranched isomers of butene, pentene, hexene, heptene, octene, nonene, decene, undecene and dodecene. The term alkene also comprises residues with more than one double bond that may be conjugated or non-conjugated, for example may comprise one double bond.

Some examples of cycloalkene are cyclopentene, cyclohexene, methylcyclopentene, dimethylcyclopentene and methylcyclohexene. Cycloalkene may comprise more than one double bond that may be conjugated or non-conjugated, for example may comprise one double bond.

In the definitions the term alkyl comprises within the given limits of carbon atoms, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, 2-methylheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl or dodecyl.

Examples of alkenyl are within the given limits of carbon atoms vinyl, allyl, and the branched and unbranched isomers of butenyl, pentenyl, hexenyl, heptenyl, octenyl, nonenyl, decenyl, undecenyl and dodecenyl. The term alkenyl also comprises residues with more than one double bond that may be conjugated or non-conjugated, for example may comprise one double bond.

Examples of alkylene are within the given limits of carbon atoms branched and unbranched isomers of vinylene, allylene, butylene, pentylene, hexylene, heptylene, octylene, nonylene, decylene, undecylene and dodecylene.

Some examples of cycloalkyl are cyclopentyl, cyclohexyl, methylcyclopentyl, dimethylcyclopentyl and methylcyclohexyl.

Some examples of cycloalkenyl are cyclopentenyl, cyclohexenyl, methylcyclopentenyl, dimethylcyclopentenyl and methylcyclohexenyl. Cycloalkenyl may comprise more than one double bond that may be conjugated or non-conjugated, for example may comprise one double bond.

Aryl is for example phenyl or naphthyl.

The term alkoxy may comprise within the limits of the given number of carbon atoms, for example methoxy and ethoxy and the branched and unbranched isomers of propoxy, butoxy, pentyloxy, hexyloxy, heptyloxy, octyloxy, nonyloxy, decyloxy, undecyloxy, dodecyloxy, tridecyloxy, tetradecyloxy, pentadecyloxy, hexadecyloxy, heptadecyloxy and octadecyloxy.

The term halogen may comprises chlorine, bromine and iodine; for example halogen is chlorine except in formula (203).

This invention also relates to the use of at least one compound or a mixture of compounds according to this invention as a stabilizer for an organic polymer against degradation by light, oxygen and/or heat or as flame retardant for an organic polymer.

For instance, this invention pertains to the use of at least one compound according to this invention as a stabilizer for an organic polymer against degradation by light, oxygen and/or heat or as flame retardant for an organic polymer.

For example, this invention pertains to the use of a mixture of compounds according to this invention as a stabilizer for an organic polymer against degradation by light, oxygen and/or heat or as flame retardant for an organic polymer.

This invention also relates to a process for flame retarding an organic polymer or stabilizing an organic polymer against degradation by light, oxygen and/or heat, which process comprises applying to or incorporating into said polymer at least one compound or a mixture of compounds according to this invention.

For instance, this invention pertains to a process for flame retarding an organic polymer or stabilizing an organic polymer against degradation by light, oxygen and/or heat, which process comprises applying to or incorporating into said polymer at least one compound according to this invention.

For example, this invention pertains to a process for flame retarding an organic polymer or stabilizing an organic polymer against degradation by light, oxygen and/or heat, which process comprises applying to or incorporating into said polymer a mixture of compounds according to this invention.

This invention further pertains to compositions comprising

A) an organic polymer which is sensitive to oxidative, thermal and/or actinic degradation, and
B) at least one compound or a mixture of compounds according to this invention.

Of interest are natural, semi-synthetic or synthetic organic polymers, especially a polyolefin or a polyolefin copolymer, for example a polyolefin.

Examples of polymers which can be protected with the compounds according to this invention are the following:

1. Polymers of monoolefins and diolefins, for example polypropylene, polyisobutylene, polybut-1-ene, poly-4-methylpent-1-ene, polyisoprene or polybutadiene, as well as polymers of cycloolefins, for instance of cyclopentene or norbornene, polyethylene (which optionally can be crosslinked), for example high density polyethylene (HDPE), high density and high molecular weight polyethylene (HDPE-HMW), high density and ultrahigh molecular weight poly-ethylene (HDPE-UHMW), medium density polyethylene (MDPE), low density polyethylene (LDPE), linear low density polyethylene (LLDPE), (VLDPE) and (ULDPE).

Polyolefins, i.e. the polymers of monoolefins exemplified in the preceding paragraph, preferably polyethylene and polypropylene, can be prepared by different, and especially by the following, methods:
a) radical polymerisation (normally under high pressure and at elevated temperature).
b) catalytic polymerisation using a catalyst that normally contains one or more than one metal of groups IVb, Vb, VIb or VIII of the Periodic Table. These metals usually have one or more than one ligand, typically oxides, halides, alcoholates, esters, ethers, amines, alkyls, alkenyls and/or aryls that may be either π- or σ-coordinated. These metal complexes may be in the free form or fixed on substrates, typically on activated magnesium chloride, titanium(III) chloride, alumina or silicon oxide. These catalysts may be soluble or insoluble in the polymerisation medium. The catalysts can be used by themselves in the polymerisation or further activators may be used, typically metal alkyls, metal hydrides, metal alkyl halides, metal alkyl oxides or metal alkyloxanes, said metals being elements of groups Ia, IIa and/or IIIa of the Periodic Table. The activators may be modified conveniently with further ester, ether, amine or silyl ether groups. These catalyst systems are usually termed Phillips, Standard Oil Indiana, Ziegler (-Natta), TNZ (DuPont), metallocene or single site catalysts (SSC).

2. Mixtures of the polymers mentioned under 1), for example mixtures of polypropylene with polyisobutylene, polypropylene with polyethylene (for example PP/HDPE, PP/LDPE) and mixtures of different types of polyethylene (for example LDPE/HDPE).

3. Copolymers of monoolefins and diolefins with each other or with other vinyl monomers, for example ethylene/propylene copolymers, linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, propylene/butadiene copolymers, isobutylene/isoprene copolymers, ethylene/alkyl acrylate copolymers, ethylene/alkyl methacrylate copolymers, ethylene/vinyl acetate copolymers and their copolymers with carbon monoxide or ethylene/acrylic acid copolymers and their salts (ionomers) as well as terpolymers of ethylene with propylene and a diene such as hexadiene, dicyclopentadiene or ethylidene-norbornene; and mixtures of such copolymers with one another and with polymers mentioned in 1) above, for example polypropylene/ethylene-propylene copolymers, LDPE/ethylene-vinyl acetate copolymers (EVA), LDPE/ethylene-acrylic acid copolymers (EAA), LLDPE/EVA, LLDPE/EAA and alternating or random polyalkylene/carbon monoxide copolymers and mixtures thereof with other polymers, for example polyamides.

4. Hydrocarbon resins (for example C₅-C₉) including hydrogenated modifications thereof (e.g. tackifiers) and mixtures of polyalkylenes and starch.

5. Polystyrene, poly(p-methylstyrene), poly(α-methylstyrene).

6. Copolymers of styrene or α-methylstyrene with dienes or acrylic derivatives, for example styrene/butadiene, styrene/acrylonitrile, styrene/alkyl methacrylate, styrene/butadiene/alkyl acrylate, styrene/butadiene/alkyl methacrylate, styrene/maleic anhydride, styrene/acrylonitrile/methyl acrylate; mixtures of high impact strength of styrene copolymers and another polymer, for example a polyacrylate, a diene polymer or an ethylene/propylene/diene terpolymer; and block copolymers of styrene such as styrene/butadiene/styrene, styrene/isoprene/styrene, styrene/ethylene/butylene/styrene or styrene/ethylene/propylene/styrene.

7. Graft copolymers of styrene or α-methylstyrene, for example styrene on polybutadiene, styrene on polybutadiene-styrene or polybutadiene-acrylonitrile copolymers; styrene and acrylonitrile (or methacrylonitrile) on polybutadiene; styrene, acrylonitrile and methyl meth-acrylate on polybutadiene; styrene and maleic anhydride on polybutadiene; styrene, acrylonitrile and maleic anhydride or maleimide on polybutadiene; styrene and maleimide on poly-butadiene; styrene and alkyl acrylates or methacrylates on polybutadiene; styrene and acrylonitrile on ethylene/propylene/diene terpolymers; styrene and acrylonitrile on polyalkyl acrylates or polyalkyl methacrylates, styrene and acrylonitrile on acrylate/butadiene copolymers, as well as mixtures thereof with the copolymers listed under 6), for example the copolymer mixtures known as ABS, MBS, ASA or AES polymers.

8. Halogen-containing polymers such as polychloroprene, chlorinated rubbers, chlorinated and brominated copolymer of isobutylene-isoprene (halobutyl rubber), chlorinated or sulfo-chlorinated polyethylene, copolymers of ethylene and chlorinated ethylene, epichlorohydrin homo- and copolymers, especially polymers of halogen-containing vinyl compounds, for example polyvinyl chloride, polyvinylidene chloride, polyvinyl fluoride, polyvinylidene fluoride, as well as copolymers thereof such as vinyl chloride/vinylidene chloride, vinyl chloride/vinyl acetate or vinylidene chloride/vinyl acetate copolymers.

9. Polymers derived from α,β-unsaturated acids and derivatives thereof such as polyacrylates and polymethacrylates; polymethyl methacrylates, polyacrylamides and polyacrylonitriles, impact-modified with butyl acrylate.

10. Copolymers of the monomers mentioned under 9) with each other or with other unsaturated monomers, for example acrylonitrile/butadiene copolymers, acrylonitrile/alkyl acrylate copolymers, acrylonitrile/alkoxyalkyl acrylate or acrylonitrile/vinyl halide copolymers or acrylonitrile/alkyl methacrylate/butadiene terpolymers.

11. Polymers derived from unsaturated alcohols and amines or the acyl derivatives or acetals thereof, for example polyvinyl alcohol, polyvinyl acetate, polyvinyl stearate, polyvinyl benzoate, polyvinyl maleate, polyvinyl butyral, polyallyl phthalate or polyallyl melamine; as well as their copolymers with olefins mentioned in 1) above.

12. Homopolymers and copolymers of cyclic ethers such as polyalkylene glycols, polyethylene oxide, polypropylene oxide or copolymers thereof with bisglycidyl ethers.

13. Polyacetals such as polyoxymethylene and those polyoxymethylenes which contain ethylene oxide as a comonomer; polyacetals modified with thermoplastic polyurethanes, acrylates or MBS.

14. Polyphenylene oxides and sulfides, and mixtures of polyphenylene oxides with styrene polymers or polyamides.

15. Polyurethanes derived from hydroxyl-terminated polyethers, polyesters or polybutadienes on the one hand and aliphatic or aromatic polyisocyanates on the other, as well as precursors thereof.

16. Polyamides and copolyamides derived from diamines and dicarboxylic acids and/or from aminocarboxylic acids or the corresponding lactams, for example polyamide 4, polyamide 6, polyamide 6/6, 6/10, 6/9, 6/12, 4/6, 12/12, polyamide 11, polyamide 12, aromatic polyamides starting from m-xylene diamine and adipic acid; polyamides prepared from hexamethylenediamine and isophthalic or/and terephthalic acid and with or without an elastomer as modifier, for example poly-2,4,4-trimethylhexamethylene terephthalamide or poly-m-phenylene isophthalamide; and also block copolymers of the aforementioned polyamides with polyolefins, olefin copolymers, ionomers or chemically bonded or grafted elastomers; or with polyethers, e.g. with polyethylene glycol, polypropylene glycol or polytetramethylene glycol; as well as polyamides or copolyamides modified with EPDM or ABS; and polyamides condensed during processing (RIM polyamide systems).

17. Polyureas, polyimides, polyamide-imides, polyetherimids, polyesterimids, polyhydantoins and polybenzimidazoles.

18. Polyesters derived from dicarboxylic acids and diols and/or from hydroxycarboxylic acids or the corresponding lactones, for example polyethylene terephthalate, polybutylene terephthalate, poly-1,4-dimethylolcyclohexane terephthalate and polyhydroxybenzoates, as well as block copolyether esters derived from hydroxyl-terminated polyethers; and also poly-esters modified with polycarbonates or MBS.

19. Polycarbonates and polyester carbonates.

20. Polysulfones, polyether sulfones and polyether ketones.

21. Crosslinked polymers derived from aldehydes on the one hand and phenols, ureas and melamines on the other hand, such as phenol/formaldehyde resins, urea/formaldehyde re-sins and melamine/formaldehyde resins.

22. Drying and non-drying alkyd resins.

23. Unsaturated polyester resins derived from copolyesters of saturated and unsaturated dicarboxylic acids with polyhydric alcohols and vinyl compounds as crosslinking agents, and also halogen-containing modifications thereof of low flammability.

24. Crosslinkable acrylic resins derived from substituted acrylates, for example epoxy acrylates, urethane acrylates or polyester acrylates.

25. Alkyd resins, polyester resins and acrylate resins crosslinked with melamine resins, urea resins, isocyanates, isocyanurates, polyisocyanates or epoxy resins.

26. Crosslinked epoxy resins derived from aliphatic, cycloaliphatic, heterocyclic or aromatic glycidyl compounds, e.g. products of diglycidyl ethers of bisphenol A and bisphenol F, which are crosslinked with customary hardeners such as anhydrides or amines, with or without accelerators.

27. Blends of the aforementioned polymers (polyblends), for example PP/EPDM, Poly-amide/EPDM or ABS, PVC/EVA, PVC/ABS, PVC/MBS, PC/ABS, PBTP/ABS, PC/ASA, PC/PBT, PVC/CPE, PVC/acrylates, POM/thermoplastic PUR, PC/thermoplastic PUR, POM/acrylate, POM/MBS, PPO/HIPS, PPO/PA 6.6 and copolymers, PA/HDPE, PA/PP, PA/PPO, PBT/PC/ABS or PBT/PET/PC.

Of particular interest is the use of compounds of formula (400) to (407) or a mixture of compounds of formulae (408) and (409) as stabilizers in synthetic organic polymers, for example a coating or a bulk polymer or article formed therefrom, especially in thermoplastic polymers and corresponding compositions as well as in coating compositions, for example in acid or metal catalyzed coating compositions. Thermoplastic polymers of most importance in present compositions are polyolefines (TPO) and their copolymers, such as listed above under items 1-3, thermoplastic polyurethan (TPU), thermoplastic rubber (TPR), polycarbonate, such as in item 19 above, and blends, such as in item 27 above. Of utmost importance are polyethylene (PE), polypropylene (PP), polycarbonate (PC) and polycarbonate blends such as PC/ABS blends.

In general the compounds of formula (400) to (407) or a mixture of compounds of formulae (408) and (409) are added to the organic polymer to be stabilized in amounts of from 0.01 to 10%, preferably from 0.01 to 5%, in particular from 0.01 to 2% (based on the organic polymer to be stabilized). Particular preference is given to the use of the compounds of formula (400) to (407) or a mixture of compounds of formulae (408) and (409) in amounts of from 0.05 to 1.5%, especially from 0.1 to 0.5%. Where compounds of formula (400) to (407) or a mixture of compounds of formulae (408) and (409) are used as flame retardants, dosages are usually higher, e.g. 0.1 to 25% by weight, mainly 0.1 to 10% by weight of the organic polymer to be stabilized and protected against inflammation.

Incorporation into the organic polymers can be effected, for example, by mixing in or applying the compounds of formula (400) to (407) or a mixture of compounds of formulae (408) and (409) and, if desired, further additives by the methods which are customary in the art. The incorporation can take place prior to or during the shaping operation, or by applying the dissolved or dispersed compound or mixture to the polymer, with or without subsequent evaporation of the solvent. In the case of elastomers, these can also be stabilized as lattices. A further possibility for incorporating the compounds of formula (400) to (407) or a mixture of compounds of formulae (408) and (409) into polymers is to add them before, during or directly after the polymerization of the corresponding monomers or prior to crosslinking. In this context the compounds of formula (400) to (407) or a mixture of compounds of formulae (408) and (409) can be added as it is or else in encapsulated form (for example in waxes, oils or polymers).

The compounds of formula (400) to (407) or a mixture of compounds of formulae (408) and (409) can also be added in the form of a masterbatch containing said compound in a concentration, for example, of from 2.5 to 25% by weight to the polymers that are to be stabilized.

The compounds of formula (400) to (407) or a mixture of compounds of formulae (408) and (409) can judiciously be incorporated by the following methods:
as emulsion or dispersion (e.g. to lattices or emulsion polymers),
as a dry mixture during the mixing in of additional components or polymer mixtures,
by direct introduction into the processing apparatus (e.g. extruders, internal mixers, etc),
as solution or melt.

Novel polymer compositions can be employed in various forms and/or processed to give various products, for example as (to give) films, fibres, tapes, moulding compositions, profiles, or as binders for coating materials, adhesives or putties.

Of interest are compositions, comprising further additives.

Of special interest are compositions, comprising as further additives phenolic and/or aminic antioxidants, hindered amine light stabilizers, UV-absorbers, phosphites, phosphonites, benzofuranones, metal stearates, metal oxides, pigments, dyes, organophsophorus compounds, hydroxylamines or flame retardants and mixtures thereof.

Examples for further additives are:

1. Antioxidants

1.1. Alkylated monophenols, for example 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethyl-phenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-di-methyl-6-(1′-methylheptadec-1′-yl)phenol, 2,4-dimethyl-6-(1′-methyltridec-1′-yl)phenol and mixtures thereof.

1.2. Alkylthiomethylphenols, for example 2,4-dioctylthiomethyl-6-tert-butylphenol, 2,4-dioctylthiomethyl-6-methylphenol, 2,4-dioctylthiomethyl-6-ethyl phenol, 2,6-di-dodecylthiomethyl-4-nonylphenol.

1.3. Hydroquinones and alkylated hydroquinones, for example 2,6-di-tert-butyl-4-methoxy-phenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis-(3,5-di-tert-butyl-4-hydroxyphenyl)adipate.

1.4. Tocopherols, for example α-tocopherol, β-tocopherol, γ-tocopherol, δ-tocopherol and mixtures thereof (Vitamin E).

1.5. Hydroxylated thiodiphenyl ethers, for example 2, 2′-thiobis(6-tert-butyl-4-methylphenol), 2,2′-thiobis(4-octyl phenol), 4,4′-thiobis(6-tert-butyl-3-methyl phenol), 4,4′-thiobis(6-tert-butyl-2-methylphenol), 4,4′-thiobis-(3,6-di-sec-amylphenol), 4,4′-bis(2,6-dimethyl-4-hydroxyphenyl)disulfide.

1.6. Alkylidenebisphenols, for example 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butyl-phenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis-(3,5-di-tert-butyl-4-hydroxyphenyl)propane, 2,2-bis-(5-tert-butyl-4-hydroxy2-methylphenyl)-4-n-dodecylmercaptobutane, 1,1,5,5-tetra-(5-tert-butyl-4-hydroxy-2-methylphenyl)pentane.

1.7. O-, N- and S-benzyl compounds, for example 3,5,3′,5′-tetra-tert-butyl-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxy-benzyl)sulfide, isooctyl-3,5-di-tert-butyl-4-hydroxybenzylmercaptoacetate.

1.8. Hydroxybenzylated malonates, for example dioctadecyl-2,2-bis-(3,5-di-tert-butyl-2-hydroxybenzyl)-malonate, di-octadecyl-2-(3-tert-butyl-4-hydroxy-5-methylbenzyl)-malonate, di-dodecylmercaptoethyl-2,2-bis-(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate.

1.9. Aromatic hydroxybenzyl compounds, for example 1,3,5-tris-(3,5-di-tert-butyl-4-hydroxy-benzyl)-2,4,6-trimethylbenzene, 1,4-bis(3,5-di-tert-butyl-4-hydroxybenzyl)-2,3,5,6-tetramethyl benzene, 2,4,6-tris(3,5-di-tert-butyl-4-hydroxybenzyl)phenol.

1.10. Triazine Compounds, for example 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris-(3,5-di-tert-butyl-4-hydroxy-benzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris(3,5-di-tert-butyl-4-hydroxyphenylpropionyl)-hexahydro-1,3,5-triazine, 1,3,5-tris(3,5-dicyclohexyl-4-hydroxybenzyl)isocyanurate.

1.11. Benzylphosphonates, for example dimethyl-2,5-di-tert-butyl-4-hydroxybenzylphosphonate, diethyl-3,5-di-tert-butyl-4-hydroxybenzylphosphonate, dioctadecyl3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid.

1.12. Acylaminophenols, for example 4-hydroxylauranilide, 4-hydroxystearanilide, octyl N-(3,5-di-tert-butyl-4-hydroxyphenyl)carbamate.

1.13. Esters of β-(3,5-di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl) isocyanurate, N,N′-bis(hydroxyethyl)oxamide, 3-thiaundecanol, 3-thiapentadecanol, trimethylhexanediol, trimethylol-propane, 4-hydroxymethyl-1-phospha-2,6,7-trioxabicyclo[2.2.2]octane.

1.14. Esters of β-(5-tert-butyl-4-hydroxy-3-methylphenyl)propionic acid with mono- or poly-hydric alcohols, e.g. with methanol, ethanol, n-octanol, i-octanol, octadecanol, 1,6-hexane-diol, 1,9-nonanediol, ethylene glycol, 1,2-propanediol, neopentyl glycol, thiodiethylene glycol, diethylene glycol, triethylene glycol, pentaerythritol, tris(hydroxyethyl) isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.15. Esters of β-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.16. Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.17. Amides of β-(3,5-di-tert-butyl-4-hydroxyphenyl)propionic acid e.g. N,N′-bis(3,5-di-tert-butyl-4-hydroxyphenylpropionyl)hexamethylenediamide, N,N′-bis(3,5-di-tert-butyl-4-hydroxy-phenylpropionyl)trimethylenediamide, N,N′-bis(3,5-di-tert-butyl-4-hydroxyphenylpropionyl)-hydrazide, N,N′-bis[2-(3-[3,5-di-tert-butyl-4-hydroxyphenyl]propionyloxy)ethyl]oxamide (Naugard®XL-1 supplied by Uniroyal).

1.18. Ascorbic acid (vitamin C)

1.19. Aminic antioxidants, for example N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclo-hexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyidiphenylamine, 4-isopropoxy-diphenylamine, N-phenyl-1-naphthylamine, N-(4-tert-octylphenyl)-1-naphthylamine, N-phenyl-2-naphthylamine, octylated diphenylamine, for example p,p′-di-tert-octyidiphenylamine, 4-n-butylaminophenol, 4-butyrylaminophenol, 4-nonanoylaminophenol, 4-dodecanoylamino-phenol, 4-octadecanoylaminophenol, bis(4-methoxyphenyl)amine, 2,6-di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyidiphenylamines, a mixture of mono- and dialkylated nonyidiphenylamines, a mixture of mono- and dialkylated dodecyldiphenylamines, a mixture of mono- and dialkylated isopropyl/isohexyldiphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octyl-phenothiazines, N-allylphenothiazin, N,N,N′,N′-tetraphenyl-1,4-diaminobut-2-ene, N,N-bis-(2,2,6,6-tetramethyl-piperid-4-yl-hexamethylenediamine, bis(2,2,6,6-tetramethylpiperid-4-yl)-sebacate, 2,2,6,6-tetramethylpiperidin-4-one, 2,2,6,6-tetramethylpiperidin-4-ol.

2. UV Absorbers and Light Stabilisers

2.1. 2-(2′-Hydroxyphenyl)benzotriazoles, for example 2-(2′-hydroxy-5′-methylphenyl)-benzo-triazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethyl butyl)phenyl)benzotriazole, 2-(3′,5′-di-tert-butyl-2′-hydroxyphenyl)-5-chloro-benzotriazole, 2-(3′-tert-butyl-2′-hydroxy-5′-methylphenyl)-5-chloro-benzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis-(α,α-dimethyl benzyl)-2′-hydroxyphenyl)benzotriazole, 2-(3′-tert-butyl-2′-hydroxy-5′-(2-octyloxycarbonylethyl)phenyl)-5-chloro-benzotriazole, 2-(3′-tert-butyl-5′-[2-(2-ethylhexyloxy)-carbonylethyl]-2′-hydroxyphenyl)-5-chloro-benzotriazole, 2-(3′-tert-butyl-2′-hydroxy-5′-(2-methoxycarbonylethyl)phenyl)-5-chloro-benzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxy-carbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylene-bis-[4-(1,1,3,3-tetramethylbutyl)-6-benzotriazole-2-ylphenol]; the transesterification product of 2-[3′-tert-butyl-5′-(2-methoxycarbonylethyl)-2′-hydroxyphenyl]-2H-benzotriazole with polyethylene glycol 300; [R—CH₂CH₂—COO—CH₂CH₂₂ where R=3′-tert-butyl-4′-hydroxy-5′-2H-benzotriazol-2-ylphenyl, 2-[2′-hydroxy-3′-(α,α-dimethyl benzyl)-5′-(1,1,3,3-tetramethylbutyl)-phenyl]benzotriazole; 2-[2′-hydroxy-3′-(1,1,3,3-tetramethylbutyl)-5′-(α,α-dimethyl benzyl)-phenyl]benzotriazole.

2.2. 2-Hydroxybenzophenones, for example the 4-hydroxy, 4-methoxy, 4-octyloxy, 4-decyloxy, 4-dodecyloxy, 4-benzyloxy, 4,2′,4′-trihydroxy and 2′-hydroxy-4,4′-dimethoxy derivatives.

2.3. Esters of substituted and unsubstituted benzoic acids, as for example 4-tertbutyl-phenyl salicylate, phenyl salicylate, octylphenyl salicylate, dibenzoyl resorcinol, bis(4-tert-butylbenzoyl)resorcinol, benzoyl resorcinol, 2,4-di-tert-butylphenyl 3,5-di-tert-butyl-4-hydroxybenzoate, hexadecyl 3,5-di-tert-butyl-4-hydroxybenzoate, octadecyl 3,5-di-tert-butyl-4-hydroxy-benzoate, 2-methyl-4,6-di-tert-butylphenyl 3,5-di-tert-butyl-4-hydroxybenzoate.

2.4. Acrylates, for example ethyl α-cyano-β,β-diphenylacrylate, isooctyl α-cyano-β,β-diphenylacrylate, methyl α-carbomethoxycinnamate, methyl α-cyano-β-methyl-p-methoxy-cinnamate, butyl α-cyano-β-methyl-p-methoxy-cinnamate, methyl α-carbomethoxy-p-methoxycinnamate and N-(β-carbomethoxy-β-cyanovinyl)-2-methylindoline.

2.5. Nickel compounds, for example nickel complexes of 2,2′-thio-bis-[4-(1,1,3,3-tetramethylbutyl)phenol], such as the 1:1 or 1:2 complex, with or without additional ligands such as n-butylamine, triethanolamine or N-cyclohexyldiethanolamine, nickel dibutyldithiocarbamate, nickel salts of the monoalkyl esters, e.g. the methyl or ethyl ester, of 4-hydroxy-3,5-di-tert-butylbenzylphosphonic acid, nickel complexes of ketoximes, e.g. of 2-hydroxy-4-methylphenyl undecylketoxime, nickel complexes of 1-phenyl-4-lauroyl-5-hydroxypyrazole, with or without additional ligands.

2.6. Further sterically hindered amines, for example bis(2,2,6,6-tetramethyl-4-piperidyl)sebacate, bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl) n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butane-tetracarboxylate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl)malonate, 3-n-octyl-7,7,9,9-tetramethyl-1,3,8-triazaspiro[4.5]decan-2,4-dione, bis(1-octyloxy-2,2,6,6-tetramethylpiperidyl)sebacate, bis(1-octyloxy-2,2,6,6-tetramethylpiperidyl)succinate, linear or cyclic condensates of N,N′-bis-(2,2,6,6-tetramethyl-4-piperidyl)hexamethylenediamine and 4-morpholino-2,6-dichloro-1,3,5-triazine, the condensate of 2-chloro-4,6-bis(4-n-butylamino-2,2,6,6-tetramethylpiperidyl)-1,3,5-triazine and 1,2-bis(3-aminopropylamino)ethane, the condensate of 2-chloro-4,6-di-(4-n-butylamino-1,2,2,6,6-pentamethylpiperidyl)-1,3,5-triazine and 1,2-bis-(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)pyrrolidin-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, a condensation product of N,N′-bis(2,2,6,6-tetramethyl-4-piperidyl)hexamethylenediamine and 4-cyclohexylamino-2,6-dichloro-1,3,5-triazine, a condensation product of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); N-(2,2,6,6-tetramethyl-4-piperidyl)-n-dodecylsuccinimid, N-(1,2,2,6,6-pentamethyl-4-piperidyl)-n-dodecylsuccinimid, 2-undecyl-7,7,9,9-tetramethyl-1-oxa-3,8-diaza-4-oxo-spiro[4,5]decane, a reaction product of 7,7,9,9-tetramethyl-2-cycloundecyl-1-oxa-3,8-diaza-4-oxospiro[4,5]decane and epichlorohydrin, 1,1-bis(1,2,2,6,6-pentamethyl-4-piperidyloxycarbonyl)-2-(4-methoxyphenyl)ethene, N,N′-bis-formyl-N,N′-bis(2,2,6,6-tetramethyl-4-piperidyl)hexamethylenediamine, diester of 4-methoxy-methylene-malonic acid with 1,2,2,6,6-pentamethyl-4-hydroxypiperidine, poly[methylpropyl-3-oxy-4-(2,2,6,6-tetramethyl-4-piperidyl)]siloxane, reaction product of maleic acid anhydride-α-olefin-copolymer with 2,2,6,6-tetramethyl-4-aminopiperidine or 1,2,2,6,6-pentamethyl-4-aminopiperidine, 2,4-bis[N-(1-cyclohexyloxy-2,2,6,6-tetramethylpiperidine-4-yl)-N-butyl-amino]-6-(2-hydroxyethyl)amino-1,3,5-triazine.

2.7. Oxamides, for example 4, 4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy-disubstituted oxanilides and mixtures of o- and p-ethoxy-disubstituted oxanilides.

2.8. 2-(2-Hydroxyphenyl)-1,3,5-triazines, for example 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis-(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxy-propoxy)phenyl]-4,6-bis(2,4-dimethyl)-1,3,5-triazine, 2-[2-hydroxy-4-(2-hydroxy-3-octyloxy-propyloxy)phenyl]-4,6-bis(2,4-dimethyl)-1,3,5-triazine, 2-[4-(dodecyloxy/tridecyloxy-2-hydroxypropoxy)-2-hydroxy-phenyl]-4,6-bis(2,4-di-methylphenyl)-1,3,5-triazine, 2-[2-hydroxy-4-(2-hydroxy-3-dodecyloxy-propoxy)phenyl]-4,6-bis(2,4-dimethylphenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxy-propoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine, 2-{2-hydroxy-4-[1-octyloxycarbonyl-ethoxy]phenyl}-4,6-bis(4-phenylphenyl)-1,3,5-triazine wherein the octyl moiety is a mixture of different isomers.

3. Metal deactivators, for example N,N′-diphenyloxamide, N-salicylal-N′-salicyloyl hydrazine, N,N′-bis(salicyloyl)hydrazine, N,N′-bis(3,5-di-tert-butyl-4-hydroxyphenylpropionyl)hydrazine, 3-salicyloylamino-1,2,4-triazole, bis(benzylidene)oxalyl dihydrazide, oxanilide, isophthaloyl dihydrazide, sebacoyl bisphenyl hydrazide, N,N′-diacetyladipoyl dihydrazide, N,N′-bis(salicyloyl)oxalyl dihydrazide, N,N′-bis(salicyloyl)thiopropionyl dihydrazide.

4. Phosphites and phosphonites, for example triphenyl phosphite, diphenyl alkyl phosphites, phenyl dialkyl phosphites, tris(nonylphenyl)phosphite, trilauryl phosphite, trioctadecyl phosphite, distearyl pentaerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,6-di-tert-butyl-4-methylphenyl)-pentaerythritol diphosphite, diisodecyloxypentaerythritol di-phosphite, bis(2,4-di-tert-butyl-6-methylphenyl)pentaerythritol diphosphite, bis(2,4,6-tris(tert-butylphenyl)pentaerythritol diphosphite, tristearyl sorbitol triphosphite, tetrakis(2,4-di-tert-butylphenyl) 4,4′-biphenylene diphosphonite, 6-isooctyloxy-2,4,8,10-tetra-tert-butyl-12H-di-benz[d,g]-1,3,2-dioxaphosphocin, 6-fluoro-2,4,8,10-tetra-tert-butyl-12-methyl-dibenz[d,g]-1,3,2-dioxaphosphocin, bis(2,4-di-tert-butyl-6-methylphenyl)methyl phosphite, bis(2,4-di-tert-butyl-6-methylphenyl)ethyl phosphite, 2,2′,2″-nitrilo[triethyltris(3,3′,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-di-yl)phosphite.

Especially preferred are the following phosphites:

Tris(2,4-di-tert-butylphenyl)phosphite (Irgafos®168, Ciba Specialty Chemicals), tris(nonylphenyl)phosphite,

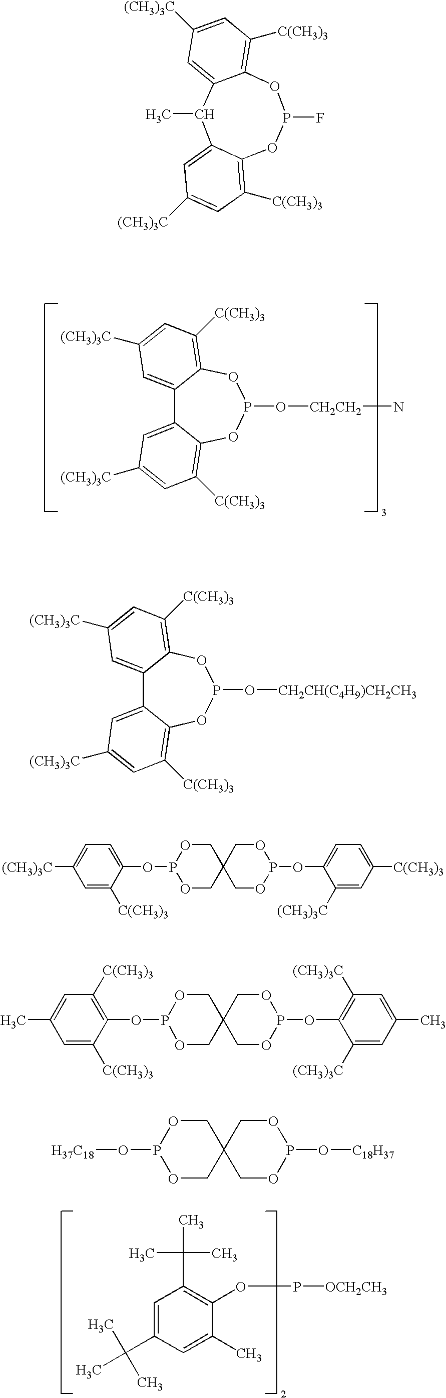

5. Hydroxylamines, for example, N,N-dibenzylhydroxylamine, N,N-diethyl hydroxylamine, N,N-dioctylhydroxylamine, N,N-dilaurylhydroxylamine, N,N-ditetradecyl hydroxylamine, N,N-dihexadecylhydroxylamine, N,N-dioctadecylhydroxylamine, N-hexadecyl-N-octadecylhydroxylamine, N-heptadecyl-N-octadecylhydroxylamine, N,N-dialkylhydroxylamine derived from hydrogenated tallow amine.

6. Nitrones, for example, N-benzyl-alpha-phenyl-nitrone, N-ethyl-alpha-methyl-nitrone, N-octyl-alpha-heptyl-nitrone, N-lauryl-alpha-undecyl-nitrone, N-tetradecyl-alpha-tridcyl-nitrone, N-hexadecyl-alpha-pentadecyl-nitrone, N-octadecyl-alpha-heptadecyl-nitrone, N-hexadecyl-alpha-heptadecyl-nitrone, N-ocatadecyl-alpha-pentadecyl-nitrone, N-heptadecyl-alpha-heptadecyl-nitrone, N-octadecyl-alpha-hexadecyl-nitrone, nitrone derived from N,N-dialkylhydroxylamine derived from hydrogenated tallow amine.

7. Thiosynergists, for example, dilauryl thiodipropionate or distearyl thiodipropionate.

8. Peroxide scavengers, for example esters of β-thiodipropionic acid, for example the lauryl, stearyl, myristyl or tridecyl esters, mercaptobenzimidazole or the zinc salt of 2-mercapto-benzimidazole, zinc dibutyldithiocarbamate, dioctadecyl disulfide, pentaerythritol tetrakis(β-dodecylmercapto)propionate.

9. Polyamide stabilisers, for example, copper salts in combination with iodides and/or phosphorus compounds and salts of divalent manganese.

10. Basic co-stabilisers, for example, melamine, polyvinylpyrrolidone, dicyandiamide, triallyl cyanurate, urea derivatives, hydrazine derivatives, amines, polyamides, polyurethanes, alkali metal salts and alkaline earth metal salts of higher fatty acids for example calcium stearate, zinc stearate, magnesium behenate, magnesium stearate, sodium ricinoleate and potassium palmitate, antimony pyrocatecholate or zinc pyrocatecholate.

11. Nucleating agents, for example, inorganic substances such as talcum, metal oxides such as titanium dioxide or magnesium oxide, phosphates, carbonates or sulfates of, preferably, alkaline earth metals; organic compounds such as mono- or polycarboxylic acids and the salts thereof, e.g. 4-tert-butylbenzoic acid, adipic acid, diphenylacetic acid, sodium succinate or sodium benzoate; polymeric compounds such as ionic copolymers (ionomers).

12. Fillers and reinforcing agents, for example, calcium carbonate, silicates, glass fibres, glass bulbs, asbestos, talc, kaolin, mica, barium sulfate, metal oxides and hydroxides, carbon black, graphite, wood flour and flours or fibers of other natural products, synthetic fibers.

13. Other additives, for example, plasticisers, lubricants, emulsifiers, pigments, rheology additives, catalysts, flow-control agents, optical brighteners, flameproofing agents, antistatic agents and blowing agents.

14. Benzofuranones and indolinones, for example those disclosed in U.S. Pat. No. 4,325,863; U.S. Pat. No. 4,338,244; U.S. Pat. No. 5,175,312; U.S. Pat. No. 5,216,052; U.S. Pat. No. 5,252,643; DE-A-4316611; DE-A-4316622; DE-A-4316876; EP-A-0589839 or EP-A-0591102 or 3-[4-(2-acetoxyethoxy)-phenyl]-5,7-di-tert-butyl-benzofuran-2-one, 5,7-di-tert-butyl-3-[4-(2-stearoyloxyethoxy)phenyl]benzofuran-2-one, 3,3′-bis[5,7-di-tert-butyl-3-(4-[2-hydroxyethoxy]phenyl)benzofuran-2-one], 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethyl phenyl)-5,7-di-tert-butyl-benzofuran-2-one, 3-(2,3-di-methylphenyl)-5,7-di-tert-butyl-benzofuran-2-one.

The conventional additives are judiciously employed in amounts of 0.1-10% by weight, for example 0.2-5% by weight, based on the organic polymer to be stabilized.

### EXAMPLES

Abbreviations for NOR building blocks:

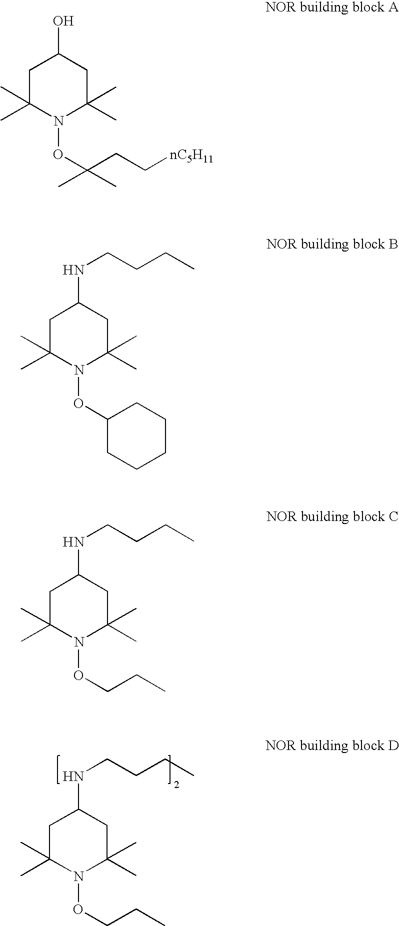

### Example 1

### Preparation of NOR Building Block A in Three Steps from Triacetonamine (TAA)

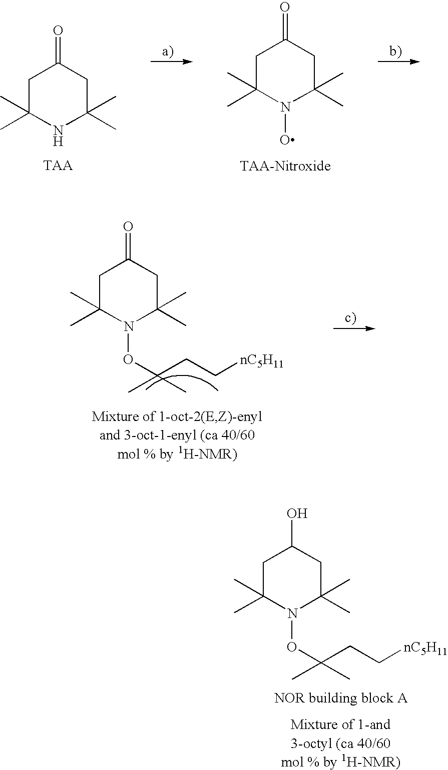

a) To a stirred mixture of 41.1 g (0.27 mol) triacetoneamine, 3.94 g (0.01 mol) sodium tungstate dihydrate and 250 ml water are added at 5° C. within 1 hour 64.9 g (0.57 mol) aqueous 30% hydrogenperoxide. The orange mixture is warmed to 25° C. and stirring is continued for 21 hours. Potassium carbonate is then added until phase separation occurs and the triacetoneamine-N-oxide extracted three times with a total of 268 g (2.39 mol) 1-octene.

b) After addition of 0.64 g (4.8 mmol) cupric chloride the combined organic phases are brought to 60° C. and 39.9 g (0.31 mol) t-butylhydroperoxide slowly dosed in. The mixture is held at 60° C. for a total of 5.6 hours. The greenish suspension is then cooled to 25° C. followed by the addition of 198 g aqueous 20% sodium sulfite. After stirring overnight the aqueous phase is split off and extracted with hexane. The combined organic phases are washed with water and concentrated on a rotary evaporator.

c) The residue is dissolved in 500 ml methanol, 9 g Ru on charcoal (5%) are added and the mixture hydrogenated at 50° C./45 bar hydrogen during 17 hours. The mixture is filtered through hyflo and the filtrate concentrated on a rotary evaporator. Distillation of the residue yields 40.4 g (53.3%) of a slightly reddish oil (bp 123° C./0.4 mbar) consisting of a mixture of 2,2,6,6-tetramethyl-1-octyloxy-piperidin-4-ol (ca 40 mol % by ¹H-NMR) and 1-(1-ethyl-hexyloxy)-2,2,6,6-tetramethyl-piperidin-4-ol (ca 60 mol % by ¹H-NMR).

Analysis required for C₁₇H₃₅NO₂ (285.47): C, 71.53%, H, 12.36%, N, 4.91%; found: C, 70.62%, H, 12.69%, N, 4.90%.

¹H-NMR (CDCl₃), δ (ppm, O—C(n)Hₓ only): 3.67 (p-like, O—C(3)H), 3.72 (t, J=ca 6.6 Hz, O—C(1)H₂), 3.95 (broad m, C(4)HOH from heterocycle).

¹³C(DEPT)-NMR (CDCl₃), δ (ppm, O—C(n)Hₓ only): 63.15 and 63.24 (C(4)HOH from heterocycle), 77.01 (O—C(1)H₂), 83.23 (O—C(3)H).

### Example 2

### Synthesis of Compound A′ by Transesterification of NOR Building Block A with Sebacic Acid Dimethylester

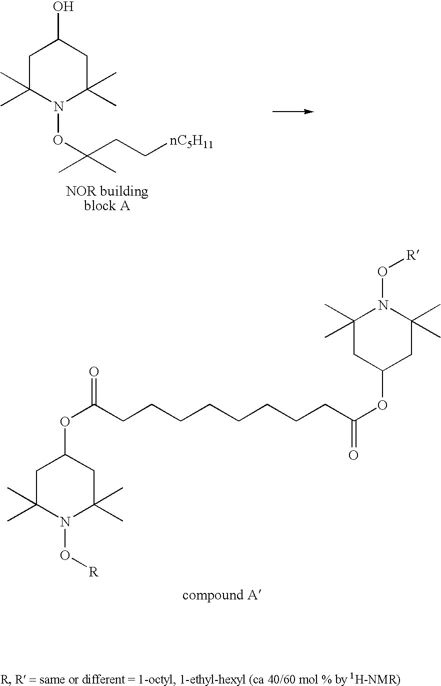

A mixture of 11.4 g (40 mmol) NOR building block A, 1.86 g (8 mmol) sebacic acid dimethylester and 0.135 g (1.6 mmol) LiOtBu are heated under vacuum (110° C., 200 mbar) during 22 hrs. The mixture is diluted with ethylacetate, washed pH neutral and the organic phase concentrated on a rotary evaporator. Flash chromatography (silica gel, hexane/ethylacetate 9/1) affords 4 g (68%) of the product as a slightly orange oil.

Analysis required for C₄₄H₈₄N₂O₆ (737.16): C, 71.69%, H, 11.49%, N, 3.80%; found: C, 71.47%, H, 11.47%, N, 3.69%.

¹H-NMR (CDCl₃), δ (ppm, O—C(n)Hₓ only): 3.67 (p-like, O—C(3)H), 3.72 (t, J=6.8 Hz, O—C(1)H₂).

¹³C(DEPT)-NMR (CDCl₃), δ (ppm, O—C(n)Hₓ only): 77.05 (O—C(1)H₂), 83.3 (O—C(3)H).

### Example 3

### Preparation of NOR Building Block B from Triacetonamine (TAA)

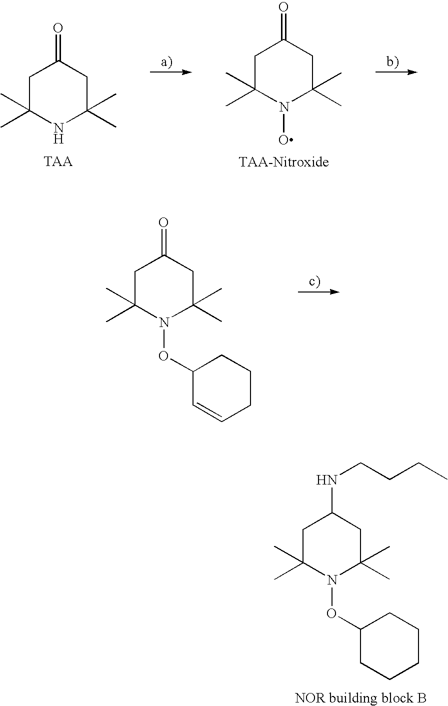

a) To a stirred mixture of 76.8 g (0.49 mol) triacetoneamine, 7.1 g (0.02 mol) sodium tungstate dihydrate and 445 ml water are added at 5° C. within 1 hour 122.5 g (1.08 mol) aqueous 30% hydrogenperoxide. The orange mixture is warmed to 25° C. and stirring is continued for 18 hours. Potassium carbonate is then added until phase separation occurs and the triacetoneamine-N-oxide extracted four times with a total of 371.5 g (4.52 mol) cyclohexene.

b) After addition of 1.01 g (4.5 mmol) cupric bromide the combined organic phases are brought to 60° C. and 49 g (0.38 mol) t-butylhydroperoxide slowly dosed in. The mixture is held at 60° C. for a total of 2.3 hours. The greenish suspension is then cooled to 25° C. followed by the addition of 280 g aqueous 20% sodium sulfite solution. After stirring 1 hour the aqueous phase is split off, the organic phase washed with water and brine and then concentrated on a rotary evaporator.

c) The residue is dissolved in 1200 ml methanol followed by the addition of 35.8 g (0.49 mol) butylamine and 16.3 g Pd on charcoal (10%). The mixture is stirred at 25° C. during 1.5 hours and then hydrogenated at 50° C./10 bar hydrogen during 1.5 hours. The mixture is filtered through Hyflo and the filtrate concentrated on a rotary evaporator. Distillation of the residue yields 112 g (73%) of a yellow to slightly orange oil (bp 120° C./0.8 mbar).

Analysis required for C₁₉H₃₈N₂O (310.53): C, 73.49%, H, 12.33%, N, 9.02%; found: C, 73.09%, H, 12.04%, N, 8.93%.

¹H-NMR (CDCl₃), δ (ppm): 0.9 (t, 3H), 1.1-1.6 (m, 24H), 1.7 (m, 4H), 2.0 (m, 2H), 2.6 (t, 2H), 2.7 (m, 1H), 3.6 (m, 1H).

¹³C-NMR (CDCl₃), δ (ppm): 14.04, 20.58, 21.17, 25.05, 25.97, 32.81, 32.84, 34.60, 46.74, 47.22, 48.20, 59.76, 81.69.

### Example 4

### Synthesis of Compound B′ by Reaction of NOR Building Block B with Cyanuric Chloride and Ethanolamine

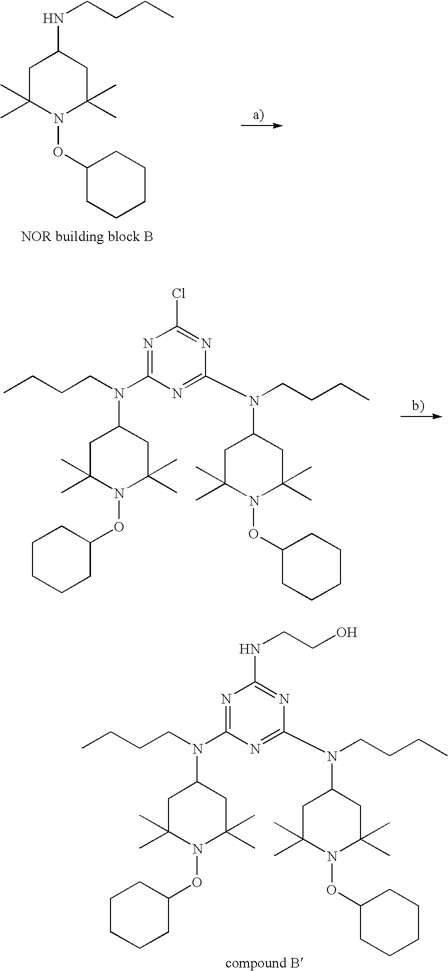

a) A solution of 6.2 g (20 mmol) NOR building block B in 10 g cyclohexane is slowly added at 25° C. to a stirred suspension of 1.9 g (10 mmol) cyanuric chloride in 8.4 g cyclohexane. Stirring is continued for 30 minutes followed by the addition of 2.7 g (20.4 mmol) aqueous 30% NaOH solution. The mixture is heated to 70° C. and stirred until the reaction is complete. The mixture is cooled down to 25° C., filtered, the aqueous phase split off and the organic phase washed with brine and concentrated on a rotary evaporator.

b) Excess ethanolamine (4 g, 65 mmol) is added and the solution heated to 110° C. Stirring is continued until the reaction is complete. The mixture is cooled down to 25° C., ethanolamine split off after addition of cyclohexane and the cyclohexane phase washed and evaporated to give a white powder.

Analysis required for C₄₃H₈₀N₈O₃ (757.17): C, 68.21%, H, 10.65%, N, 14.80%; found: C, 68.37%, H, 10.60%, N, 14.05%.

The as-prepared product exhibits higher quality compared to state-of-the-art material in terms of monomer content and transmission:

| compound B′ | HPLC [Area %]^{a)} | Transmission [%]^{b)} |
|---|---|---|
| State of the art (Tinuvin ® 152; | 53 | 85.5 |
| CAS-no. 150686-79-6)) | | |
| prepared according to Example | 42 | 76.4 |
| 4 (reactant NOR building block | | |
| B not distilled) | | |
| prepared according to Example | 80 | 88.6 |
| 4 (reactant NOR building block | | |
| B distilled) | | |

^{a)}Area of product (monomer) peak (retention time 28 min) relative to sum of peaks; conditions: AAD-0004/2 with modified column (ZORBAX Extend C-18 column, 4.6 mm × 250 mm/5 μm, AGILENT No. 770450-902; column exhibiting enhanced stability at high pH);
^{b)}425 nm, 10% w/v solutions in m-xylene.

### Example 5

### Synthesis of compound C′ by reaction of NOR building block B with cyanuric chloride and N,N′-bis(3-aminopropyl)ethylenediamine

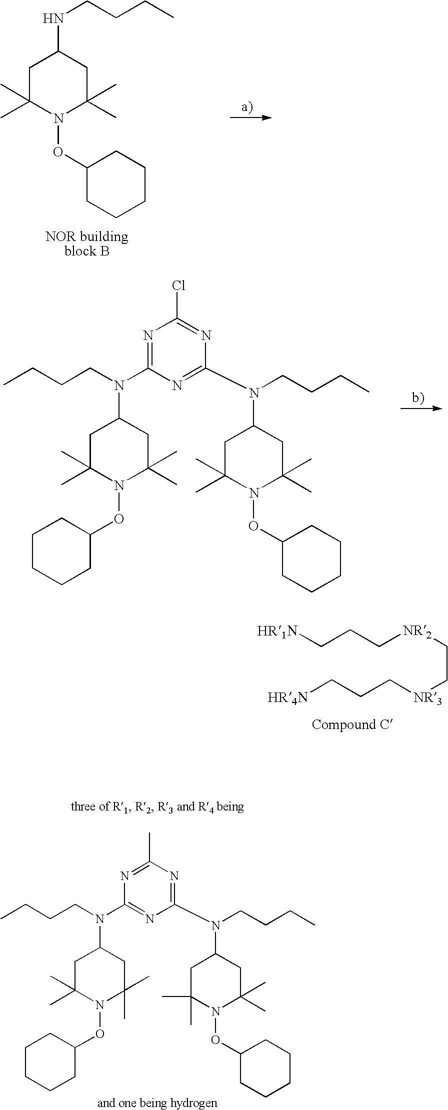

a) A solution of 6.5 g NOR building block B in 10 g cyclohexane is slowly added at 25° C. to a stirred suspension of 1.9 g (10 mmol) cyanuric chloride in 10 g cyclohexane. Stirring is continued for 30 minutes followed by the addition of 2.7 g (20.4 mmol) aqueous 30% NaOH solution. The mixture is heated to 70° C. and stirred until the reaction is complete. The mixture is cooled down to 25° C., filtered, the aqueous phase split off and the organic phase washed with brine and concentrated on a rotary evaporator.

b) A mixture of 6 g (8.2 mmol) of the above crude product, 0.47 g (2.7 mmol) N,N′-bis(3-aminopropyl)ethylenediamine and 1.7 g (8.5 mmol) aqueous 20% NaOH solution is heated in a glass pressure bottle to 125° C. during 17.5 hrs. The mixture is cooled down to 25° C., diluted with cyclohexane and the aqueous phase split off. The organic phase is brine washed and concentrated on a rotary evaporator. The crude oil is slowly added to boiling methanol, yielding a white precipitate. The suspension is treated with an Ultraturrax, filtered and the filtercake dried to yield the product as a white powder.

Analysis required for C₁₃₁H₂₄₁N₂₅O₆ (2262.51): C, 69.54%, H, 10.74%, N, 15.48%; found: C, 69.56%, H, 10.60%, N, 15.25%.

The as-prepared product exhibits higher quality compared to state-of-the-art material in terms of transmission:

| | Transmission [%]^{b} | | |
|---|---|---|---|
| Compound C′ | 425 nm | 450 nm | 500 nm |
| State of the art | 68 | 75 | 84 |
| (Flamestab ® NOR 116; | | | |
| CAS-no. 191680-81-6) | | | |
| prepared according to | 81 | 88 | 94 |
| Example 5 | | | |

### Example 6

### Preparation of NOR Building Block C in Two Steps from Triacetonamine N-Oxide

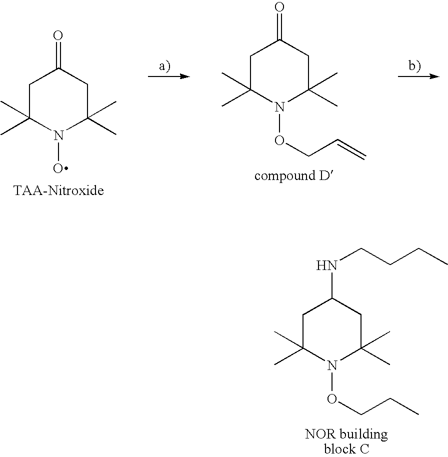

a) In a 300 ml stainless steel autoclave are added 5.01 g (29.45 mmol) triacetone amine N-oxide, 198 mg (0.9 mmol) CuBr₂ and 286 mg (0.9 mmol) Bu₄NBr. The autoclave is sealed and 38.6 g (920 mmol) of propylene are added. The reaction is heated to 70° C. (pressure ca 28 bar). When the temperature is reached, 7.6 g (58.8 mmol) of t-BuOOH (aqueous 70%) are added during 2.5 hours. The reaction is stirred for an additional 2 hours. The measured oxygen concentration of the gas phase is uncritical (<5%) throughout the reaction. Then the pressure in the autoclave is released. The autoclave is unloaded and rinsed with 50 ml of dichloromethane. GLC analysis of the reaction mixture reveals about 90% conversion. The solvents are removed from the reaction mixture and the crude product (6.9 g) purified by flash chromatography (silica gel, hexane/ethylacetate 3/1). Yield 4.1 g (66%) of a white solid (mp 50-51° C.; bp ca 80° C./1 mbar).

Analysis required for C₁₂H₂₁NO₂ (211.31): C, 68.21%, H, 10.02%, N, 6.63%; found: C, 68.76%, H, 10.15%, N, 6.55%.

¹H-NMR (400 MHz, CDCl₃), δ (ppm): 1.18 (s, 6H), 1.31 (s, 6H), 2.22 (d, J=12.8 Hz, 2H), 2.57 (d, J=12.8 Hz, 2H), 4.38 (d×t, J=5.6 Hz/1.2 Hz, 2H), 5.17 (d×q, J=10.6 Hz/1.6 Hz, 1H), 5.30 (d×q, J=17.4 Hz/1.6 Hz, 1H), 5.88-5.95 (m, 1H).

¹³C-NMR (100 MHz, CDCl₃), δ (ppm): 22.4 (2 CH₃), 32.4 (2 CH₃), 53.2 (2 CH₂), 62.9 (2 CN), 78.4 (OCH₂), 116.6 (CH₂), 133.3 (CH), 207.8 (CO).

LC/MS (m/z): 212 (MH⁺)

b) A mixture of 86.7 g (0.41 mol) compound D′, 35.2 g (0.476 mol) butylamine and 0.8 g 10% Pt on charcoal is hydrogenated over night at 80° C. and 50 bar. Filtration and evaporation of volatiles yields 104.2 g (93.9%) of a slightly yellow oil.

Analysis required for C₁₆H₃₄N₂O (270.46): C, 71.06%, H, 12.67%, N, 10.36%; found: C, 70.86%, H, 12.54%, N, 10.49%.

¹H-NMR (400 MHz, CDCl₃), δ (ppm): 0.93 (q, 6H), 1.17 (s, 6H), 1.19 (s, 6H), 1.2-1.31 (m, 2H), 1.32-1.37 (m, 2H), 1.41-1.47 (m, 2H), 1.51-1.56 (m, 2H), 1.71-1.74 (m, 2H), 2.59 (t, 2H), 2.73-2.78 (m, 1H), 3.69 (t, 2H).

¹³C(DEPT)-NMR (100 MHz, CDCl₃), δ (ppm): 10.95 (CH₃), 14.03 (CH₃), 20.6 (CH₂), 21.0 (CH₃), 21.8 (CH₂), 32.8 (CH₂), 33.3 (CH₃), 46.8 (CH₂), 48.2 (CH), 59.8 (C), 78.4 (CH₂).

### Example 7

### Preparation of NOR Building Block D in One Step

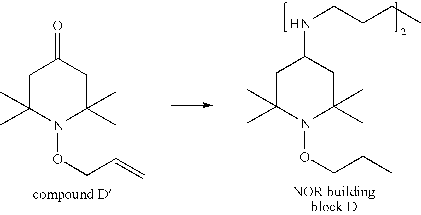

A mixture of 279 g (1.32 mol) compound D′, 71.8 g (0.6 mol) 1,6-diaminohexane, 420 ml ethanol and 1.2 g 10% Pt on charcoal is hydrogenated over night at 100° C. and 50 bar. The reaction mixture is filtered and volatiles evaporated to yield 315.5 g (100%) of a slightly orange, viscous oil.

Analysis required for C₃₀H₆₂N₄O₂ (510.85): C, 70.54%, H, 12.23%, N, 10.97%; found: C, 70.47%, H, 12.39%, N, 10.94%.

¹H-NMR (400 MHz, CDCl₃), δ (ppm): 0.95 (t, 6H), 1.15 (s, 12H), 1.18 (s, 12H), 1.20-1.26 (m, 4H), 1.34-1.36 (br m, 4H), 1.46-1.49 (m, 4H), 1.51-1.58 (m, 4H), 1.72-1.75 (m, 4H), 2.60 (t, 4H), 2.75-2.80 (m, 2H), 3.71 (t, 4H).

¹³C(DEPT)-NMR (100 MHz, CDCl₃), δ (ppm): 10.95 (CH₃), 20.95 (CH₃), 21.96 (CH₂), 27.38 (CH₂), 30.57 (CH₂), 33.24 (CH₃), 46.63 (CH₂), 46.98 (CH₂), 48.14 (CH), 59.73 (C), 78.45 (CH₂).

### Example 8

### Reaction of Cyanuric Chloride with NOR Building Blocks C and D

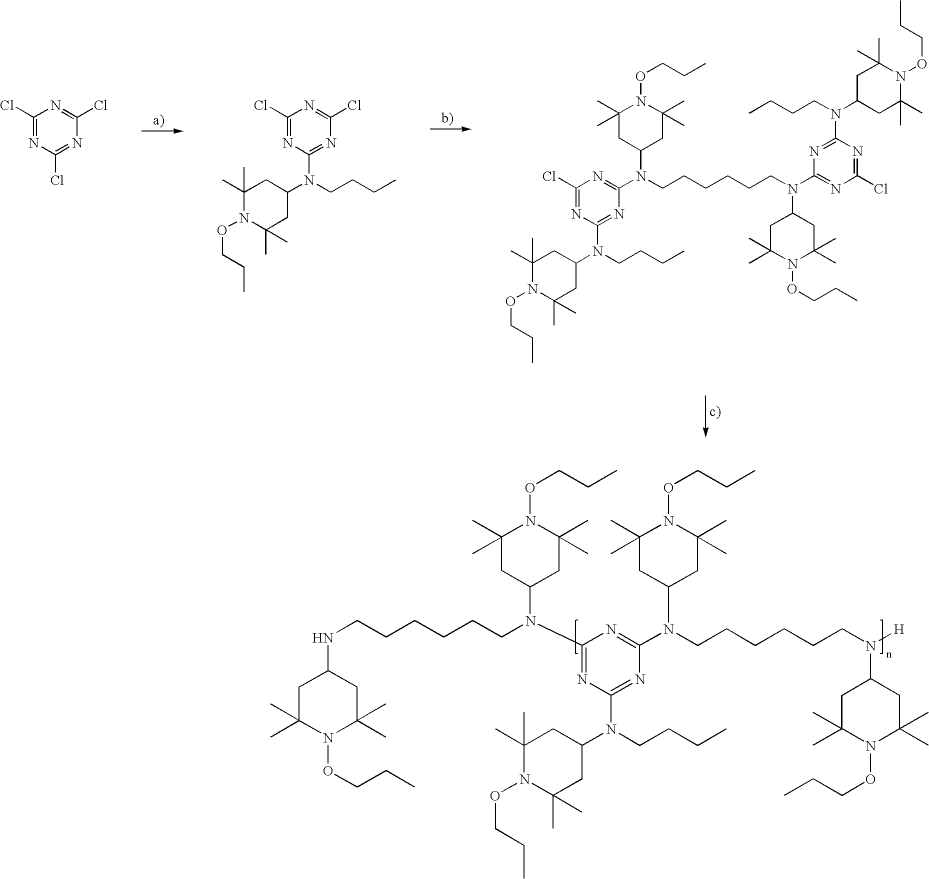

a) To a suspension of 24 g (0.13 mol) cyanuric chloride in 125 ml xylene are slowly added at 5-10° C. 35.2 g (0.13 mol) NOR building block C. The mixture is allowed to warm up to 40° C. followed by the addition of 29 g (0.145 mol) NaOH (aqueous 20%). After stirring for one hour at 40° C., a sample is withdrawn and analyzed. GLC indicates >90% conversion. The structure is confirmed by NMR.

b) The aqueous phase is split off and the organic phase heated to 70° C. followed by the slow addition of 33.2 g (0.065 mol) melted NOR building block D and 33 g water. After addition of NaOH (aqueous 30%, 20 g, 0.15 mol) the mixture is brought to 80° C. where it is left for one hour. The structure is confirmed by NMR.

c) The hot aqueous phase is split off. The organic phase is cooled down to 25° C. and transferred into an autoclave. After addition of 66.4 g (0.13 mol) NOR building block D and 28.6 g (0.143 mol) NaOH (aqueous 20%) the autoclave is sealed and heated to 175° C. where it is left for 4 hours. After cooling down to 25° C. the autoclave is unloaded and the aqueous phase split off (at 80° C.). The structure is confirmed by NMR. Mn/Mw (GPC) 1700/3300-1900/3800. Amount of residual NOR building block D ca 10% (area %).

d) Further reaction with 2-chloro-4,6-bis(dibutylamino)-s-triazine yields:

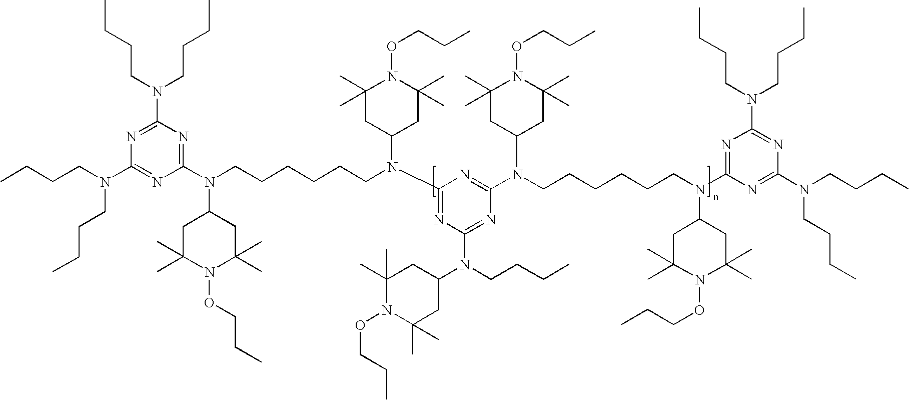

### Example 9

### Preparation of Compound D′

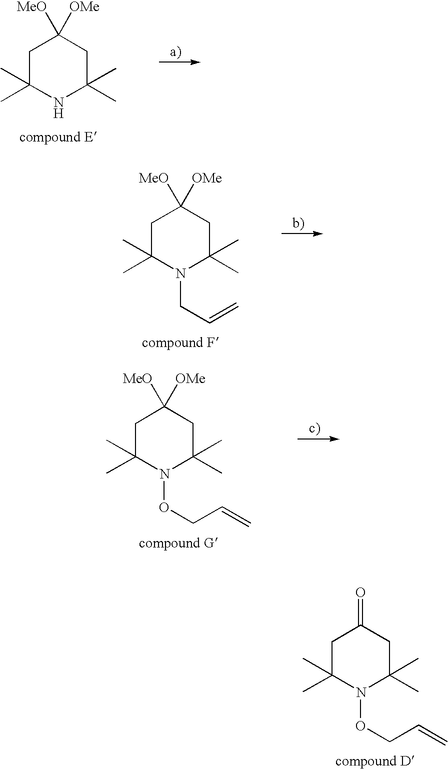

a) A mixture of 15.1 g (75 mmol) compound E′ (synthesized according to EP748849, Rhone-Poulenc), 20 g (150 mmol) NaOH (aqueous 30%), 1.27 g (3.7 mmol) Bu₄NHSO₄ and 18.3 g (151.3 mmol) allylbromide is stirred at 90° C. during 24 hours (95% conversion by GLC). The mixture is cooled down to 25° C. followed by the addition of toluene (20 ml). The aqueous phase is split off and the organic phase concentrated on a rotary evaporator. Distillation of the residue affords 8.1 g (45%) of a slightly yellow oil.

Analysis required for C₁₄H₂₇NO₂ (241.38): C, 69.67%, H, 11.27%, N, 5.80%; found: C, 69.62%, H, 10.95%, N, 5.77%.

¹H-NMR (300 MHz, CDCl₃), δ (ppm): 1.10 (s, 12H), 1.72 (s, 4H), 3.18-3.21 (m, 2H), 3.19 (s, 6H), 4.94 (d×q, J=10.2 Hz/2 Hz, 1H), 5.16 (d×q, J=17.1 Hz/2 Hz, 1H), 5.80-5.92 (m, 1H).

b) To a mixture of 23 g (95 mmol) compound F′ and 19.28 g (182 mmol) Na₂CO₃ in 200 ml toluene is added at −5° C. 20.48 g (105 mmol) AcOOH (39% in AcOH) during 40 minutes. The mixture is stirred at 0° C. (6 hours; 83% conversion by GLC) and then filtered. The filtrate is washed with NaOH 1M (3×20 ml) and brine (3×20 ml). The organic phase is dried (Na₂SO₄), filtered and the solvent evaporated. The residue is flash-filtrated over silicagel (hexane) to afford, after evaporation of the solvent, 15 g (61%) of a yellow liquid.

Analysis required for C₁₄H₂₇NO₃ (257.38): C, 65.33%, H, 10.57%, N, 5.44%; found: C, 65.48%, H, 10.80%, N, 5.33%.

¹H-NMR (400 MHz, CDCl₃), δ (ppm): 1.10 (s, 6H), 1.27 (s, 6H), 1.59 (d, J=13 Hz, 2H), 1.94 (d, J=13 Hz, 2H), 3.17 (s, 6H), 4.30 (d×t, J=5.2 Hz/1.6 Hz, 2H), 5.14 (d×q, J=10.4 Hz/1.6 Hz, 1H), 5.29 (d×q, J=17.4 Hz/1.6 Hz, 1H), 5.86-5.96 (m, 1H).

c) A solution of 1 g (3.9 mmol) compound G′, 1 g water and one drop (pasteur pipette) HCl (aqueous 32%) in 6 ml THF is stirred at 25° C. After 4 hours (97% conversion by GLC) NaHCO₃ is added, the mixture filtrated and the filtrate concentrated on a rotary evaporator. The residue is extracted with hexane to afford, after evaporation of the solvent, 0.62 g (75%) of a white solid.

¹H-NMR: same as in Example 6a.

### Example 10

### Preparation of Compound D′ Using an Oxidation Methodology Published by H. Adam et al., J. Org. Chem. 61, 1467-1472 (1996)

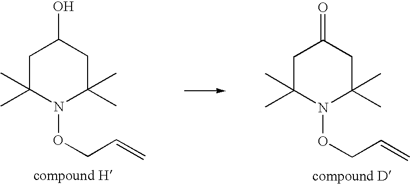

To a mixture of 5.05 g (23.7 mmol) compound H′ (synthesized according to Ciba patent DE19907945), 1.03 g (2.7 mmol) Zr(OtBu)₄ and 9.5 g activated molecular sieve (4 Å) in 45 ml toluene are slowly added at 25° C. 10.66 g (47.3 mmol) t-BuOOH (40% in cyclohexane). The mixture is stirred at 25° C. for 24 hours (86% conversion by GLC) and then washed with saturated aqueous sodium potassium tartrate and brine. The aqueous phase is split off and the organic phase dried (Na₂SO₄). Evaporation of the solvent yields 3 g of a slightly orange solid, which is analyzed by 400 MHz ¹H-NMR adding 4,4′-di-tert-butylbiphenyl as internal standard. Yield calculated based on NO—CH₂CH═CH₂ (δ=4.38 ppm) 2.1 g (42%).

¹H-NMR: same as in Example 6a.

### Example 11

### Preparation of Compound G′

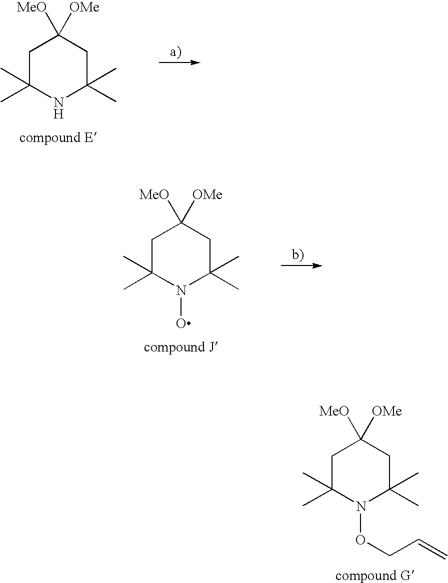

a) To a mixture of 2.1 g (10 mmol) compound E′ and 0.16 g (0.48 mmol) Na₂WO₄x2H₂O in 10 ml water are slowly added at 5° C. 2.7 g (24 mmol) H₂O₂ (aqueous 30%). The mixture is stirred at 25° C. until the starting material had disappeared (6 hours). Diethylether (20 ml) is added and the aqueous phase saturated with K₂CO₃. The aqueous phase is split off and washed with diethylether. The organic phases are combined, the solvent evaporated and the residue dried on an oil pump to afford 2.15 g (99%) of a red liquid.

Analysis required for C₁₁H₂₂NO₃ (216.30): C, 61.08%, H, 10.25%, N, 6.48%; found: C, 61.03%, H, 10.08%, N, 6.39%.

b) Compound G′ is synthesized in analogy to example 6a from 6.35 g (29.4 mmol) compound J′, 38.6 g (920 mmol) propylene, 0.328 g (0.9 mmol) Bu₄NI and 7.6 g (58.8 mmol) t-BuOOH (aqueous 70%). GLC analysis of the reaction mixture reveals about 50% conversion. Non-reacted CG43-0819 is separated off by flash-chromatography (silica gel, hexane/ethylacetate 8/2) and the dried residue analyzed by 400 MHz ¹H-NMR adding 4,4′-di-tert-butylbiphenyl as internal standard. Yield calculated based on NO—CH₂CH═CH₂ (δ=4.30 ppm) 1.5 g (20%).

¹H-NMR: same as in Example 9b. LC/MS (m/z): 258 (MH⁺)

### Example 12

### Preparation of Compound K′ by Oxidation of Chimassorb® 2020

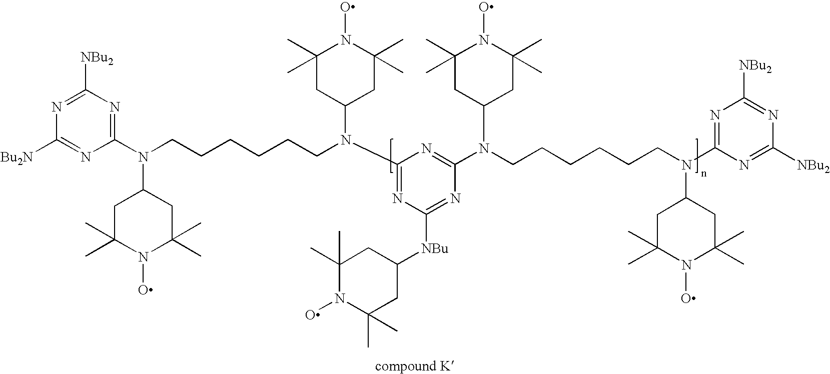

To a mixture of 20 g Chimassorb® 2020 (commercially available from Ciba Specialty Chemicals; Mₙ by GPC: 2819 g/mol, ca. 3.5 meq NH/g; CAS-no. 192268-64-7) and 35.5 g (336.5 mmol) Na₂CO₃ in 40 ml of CH₂Cl₂ are slowly added at −5° C. 26.6 g (136.5 mmol) of AcOOH (39% in AcOH). The mixture is kept stirable by concomitant, slow addition of a total of 90 ml of water. The mixture is then stirred overnight at 20° C. and the organic phase split off. The aqueous phase is extracted with CH₂Cl₂ and the combined organic phases washed with NaOH and brine, dried over MgSO₄ and the solvent evaporated to afford 18 g of a red powder.

Analysis: found C, 65.17%, H, 10.00%, N, 16.84%, 06.64%.

### Example 13

### Preparation of compound L′ from compound K′ via t-BuOOH hydrogen abstraction from propylene

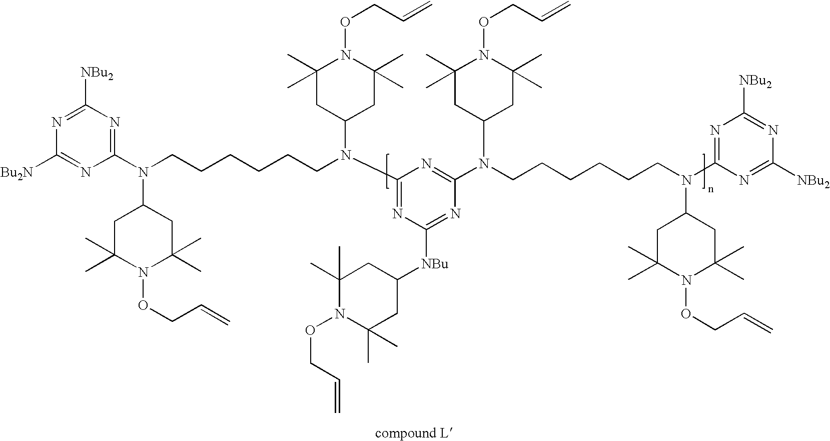

An autoclave is charged with 2.23 g compound K′ (ca 3.3 meq NO/g), 0.081 g (0.22 mmol) Bu₄NI and 10 ml chlororbenzene. The autoclave is sealed followed by the addition of 19.3 g (458.6 mmol) propylene. The system is then brought to 70° C. (ca 22 bar), whereafter 2.85 g (22.1 mmol) t-BuOOH (aqueous 70%) are slowly pumped in during 2 hours. The reaction mixture is held for another 30 minutes at 70° C. and then cooled to 25° C. (ca 10 bar). Pressure is released and the autoclave uncharged. Volatiles are removed on a rotary evaporator and the residue dried, affording compound L′ as yellowish powder.

¹H-NMR (300 MHz, CDCl₃), δ (ppm, NO—CH₂CHCH₂ only): 4.3 (br s)

1. A process for the preparation of a sterically hindered amine ether of the formula (100) 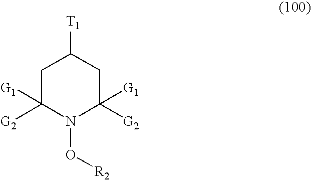
wherein
G₁ and G₂ are independently C₁-C₄alkyl;
R₂ is C₃-C₁₈alkyl or C₅-C₁₂cycloalkyl;
T₁ is hydroxy, —NT₂T₃, —OT₂₂, T₂₀ or a group of formula (102);
T₂ is hydrogen, C₅-C₁₂cycloalkyl or R₄₂; or T₂ is R₄₂ substituted by C₁-C₁₈alkoxy, aryl, hydroxy, carboxy, —CO—O—R₄₂, or —O—CO—R₂;
T₃ is hydrogen, C₅-C₁₂cycloalkyl, R₄₂, aryl, -Q-NHT₂ or -Q-NT₂T₂₁; or T₃ is R₄₂ substituted by C₁-C₁₈alkoxy, aryl, hydroxy, carboxy, —CO—O—R₄₂, or —O—CO—R₄₂; or T₃ is aryl substituted by C₁-C₁₈alkoxy, aryl, hydroxy, carboxy, —CO—O—R₄₂, —O—CO—R₄₂ or halogen;
or T₂ and T₃ form together C₄-C₁₁alkylene or C₄-C₁₁alkylene substituted by C₁-C₁₈alkoxy, aryl, hydroxy, carboxy, —CO—O—R₄₂, or —O—CO—R₄₂;
with the proviso that T₂ and T₃ are not benzyl;
R₄₂ is C₁-C₁₈alkyl;
Q is C₂-C₁₈alkylene, C₅-C₁₂cycloalkylene or phenylene;
T₂₂ is —(CO)—(C₁-C₁₆alkylene)_{0 or 1}-(CO)—O-T₂₁; 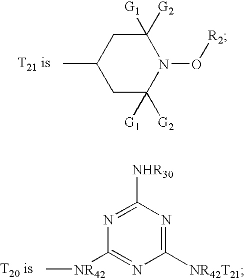
R₃₀ is R₄₂ or R₄₂ substituted by hydroxy; or R₃₀ is —(CH₂)ₙ—NT₂₃-(CH₂)ₚ—NT₂₃-(CH₂)ₙ—NHT₂₃ with one T₂₃ substituent being hydrogen and two T₂₃ substituents being 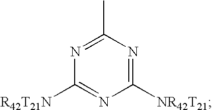
n is 1 to 4;
p is 1 to 3;
the group of formula (102) is 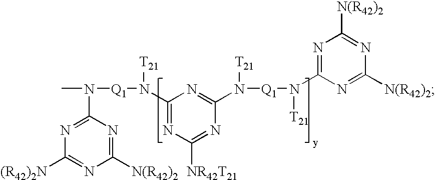
y is 2 to 20;
which comprises transforming a compound of formula (101), 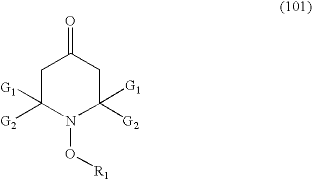
wherein
R₁ is C₃-C₁₈alkenyl or C₅-C₁₂cycloalkenyl,
in one reaction step in the presence of hydrogen and a catalyst into a compound of formula (100) wherein T₁ is hydroxy or —NT₂T₃;
whereby for obtaining compounds with T₁=—NT₂T₃ the transformation is performed in the presence of an amine of formula HNT₂T₃₀;
T₃₀ is hydrogen, C₅-C₁₂cycloalkyl, R₄₂, aryl or -Q-NHT₂; or T₃₀ is R₄₂ substituted by C₁-C₁₈alkoxy, aryl, hydroxy, carboxy, —CO—O—R₄₂, or —O—CO—R₄₂; or T₃₀ is aryl substituted by C₁-C₁₈alkoxy, aryl, hydroxy, carboxy, —CO—O—R₄₂, —O—CO—R₄₂ or halogen;
or T₂ and T₃₀ form together C₄-C₁₁alkylene or C₄-C₁₁alkylene substituted by C₁-C₁₈alkoxy, aryl, hydroxy, carboxy, —CO—O—R₄₂, or —O—CO—R₄₂;
with the proviso that T₃₀ is not benzyl;
and for obtaining a compound of formula (100) with T₁=—OT₂₂, reacting a compound of formula (100) with T₁=hydroxy with an HOOC—(C₁-C₁₆alkylene)_{0 or 1}-COOH or a halide thereof or a methyl ester thereof;
for obtaining a compound of formula (100) with T₁=T₂₀, and R₃₀=R₄₂ substituted by hydroxy, reacting a compound of formula (100) with T₁=—NT₂T₃, T₂=H, T₃=R₄₂ with a cyanuric halide to yield a compound of formula (103), which is subsequently reacted with R₄₂NH₂ or hydroxy-substituted R₄₂NH₂; 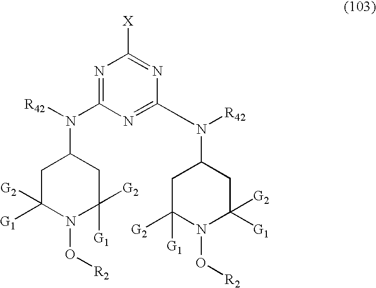
wherein
X is halogen;
for obtaining a compound of formula (100) with T₁=T₂₀ and R₃₀=—(CH₂)ₙ—NT₂₃-(CH₂)ₚ—NT₂₃-(CH₂)ₙ—NHT₂₃,
a compound of formula (103) is reacted with H₂N—(CH₂)ₙ—NH—(CH₂)ₚ—NH—(CH₂)ₙ—NH₂; and
for obtaining a compound of formula (100) with T₁=group of formula (102), reacting a compound of formula (100) with T₁=—NT₂T₃, T₂=H, T₃=R₄₂, with a cyanuric halide to yield a compound of formula (104),
which is subsequently reacted with a compound of formula (100) with T₁=—NT₂T₃, T₂=H, T₃=-Q-NHT₂₁, to yield a compound of formula (105),
which is subsequently reacted with a compound of formula (100) with T₁=—NT₂T₃, T₂=H, T₃=-Q-NHT₂₁, to yield a compound of formula (106),
which is subsequently reacted with a compound 2-X-4,6-bis((R₄₂)₂amino)-s-triazine

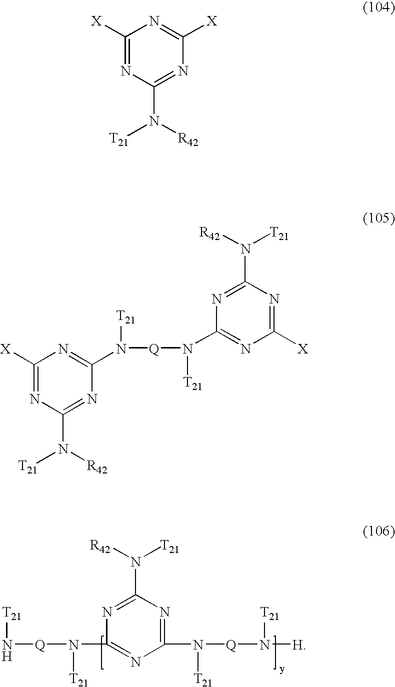

2. A process according to claim 1, wherein the catalyst is Ru, Pt or Pd on charcoal or Raney-Ni.

3. A process according to claim 1, wherein the transformation is carried out at a temperature of 35-120° C. and a hydrogen pressure of 6-100 bar.

4. A process according to claim 3, wherein the temperature is 45-110° C. and the hydrogen pressure is 8-60 bar.

5. A process according to claim 1, wherein
R₂ is C₃-C₁₀alkyl or C₅-C₇cycloalkyl;
T₂ is hydrogen;
T₃ is R₄₂, -Q-NHT₂ or -Q-NT₂T₂₁;
R₄₂ is C₁-C₈alkyl;
Q is C₂-C₈alkylene;
T₂₂ is —(CO)—C₄-C₁₀alkylene-(CO)—O-T₂₁;
n is 2 to 4;
y is 2 to 10;
R₁ is C₃-C₁₀alkenyl or C₅-C₇cycloalkenyl and
X is chlorine, bromine or iodine.

6. A process according to claim 1, wherein the compound of formula (101) is obtained by reacting a compound of formula (200) with a C₃-C₁₈alkene or C₅-C₁₂cycloalkene

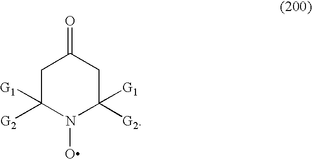

7. A process according to claim 6, wherein the compound of formula (200) is obtained by oxidizing a compound of formula (201)

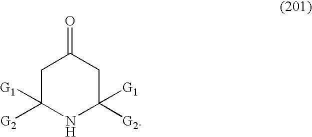

8. A process according to claim 7, which comprises the conversion of a compound of formula (201) to a compound of formula (100) without the isolation of the intermediate products.

9. A process according to claim 6, which comprises the conversion of a compound of formula (200) to a compound of formula (100) without the isolation of the intermediate products.

10. A process according to claim 1, wherein the compound of formula (101) with R₁ being the group 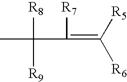
wherein
R₅, R₆, R₇, R₈ and R₉, independently of each other, are H, C₁-C₈alkyl, C₂-C₈alkenyl; and R₇ and R₈ together may also form a chemical bond;
is obtained by reacting a compound of formula (202) with a compound of formula (203), 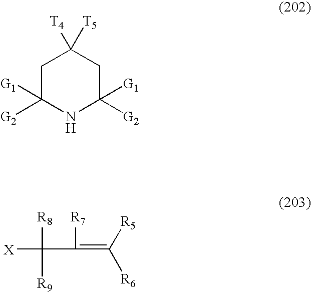
wherein T₄ and T₅ are independently C₁-C₁₈alkoxy; or T₄ is hydroxy and T₅ is hydrogen;
X is halogen;
affording a compound of formula (204); 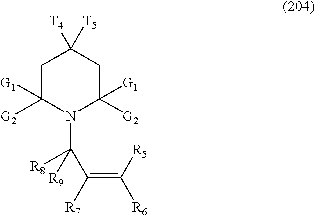
oxidizing the compound of formula (204) in the presence of oxygen, peroxides, permanganates or chlorates affords a compound of formula (205); and 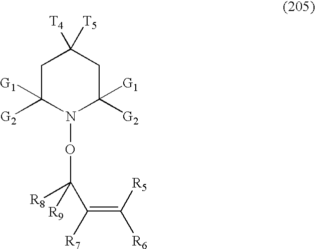
deacetalising the compound of formula (205) with T₄ and T₅ being independently C₁-C₁₈alkoxy or oxidizing the compound of formula (205) with T₄=hydroxy and T₅=hydrogen.

11. A process for the preparation of a compound of formula (300) 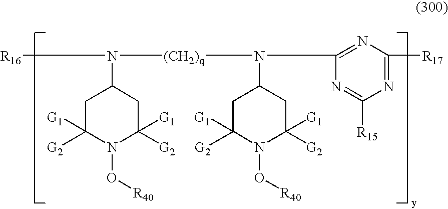
wherein
G₁ and G₂ are independently C₁-C₄alkyl;
R₄₀ is propyl or 2-propenyl;
y is 2 to 20;
q is 2 to 8;
R₁₅ is morpholino, piperidino, 1-piperizinyl, alkylamino of 1 to 8 carbon atoms, —N(C₁-C₈alkyl)T₁₀, or —N(alkyl)₂ of 2 to 16 carbon atoms, 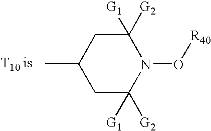
R₁₆ is hydrogen, C₂-C₄acyl, carbamoyl substituted by C₁-C₄alkyl, s-triazinyl substituted once by chlorine and once by R₁₅, or s-triazinyl substituted twice by R₁₅ with the condition that the two R₁₅ substituents may be different;
R₁₇ is chlorine, amino substituted by C₁-C₈alkyl or by T₁₀, —N(C₁-C₈alkyl)T₁₀, —N(alkyl)₂ of 2 to 16 carbon atoms, or the group T₁₃ 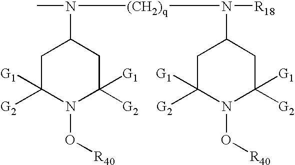
R₁₈ is hydrogen, C₂-C₄acyl, carbamoyl substituted by C₁-C₄alkyl, s-triazinyl substituted twice by —N(alkyl)₂ of 2 to 16 carbon atoms or s-triazinyl substituted twice by —N(C₁-C₈alkyl)T₁₀;
which comprises oxidizing a compound of formula (300) wherein >N—O—R₄₀ is >N—H to a compound of formula (300) wherein —O—R₄₀ is —O, which is subsequently reacted with propene;
and optionally further hydrogenating this compound for obtaining a compound of formula (300) with R₄₀=propyl.

12. A process according to claim 1, wherein G₁ and G₂ are methyl.

13. A compound of formula (400), (401), (402), (403), (404), (405), (406) or (407) 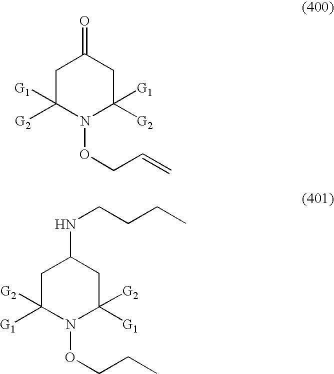
wherein G₁ and G₂ are independently C₁-C₄alkyl;
R₃₀ is C₁-C₈alkyl and
n₂ is 2 to 20;
or a mixture of compounds of formulae (408) and (409), 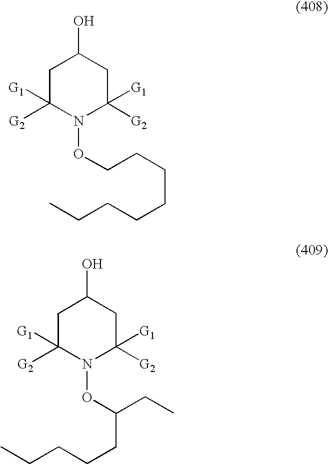
wherein G₁ and G₂ are independently C₁-C₄alkyl.

14. (canceled)

15. A compound or a mixture of compounds according to claim 13, wherein G₁ and G₂ are methyl.

16. (canceled)

17. A process for flame retarding an organic polymer or stabilizing an organic polymer against degradation by light, oxygen and/or heat, which process comprises applying to or incorporating into said polymer at least one compound or a mixture of compounds as defined in claim 13.

18. A composition comprising
A) an organic polymer which is sensitive to oxidative, thermal and/or actinic degradation, and B) at least one compound or a mixture of compounds as defined in claim 13.

19. A composition according to claim 18, comprising further additives.

20. A composition according to claim 19, comprising as further additives phenolic or aminic antioxidants, hindered amine light stabilizers, UV-absorbers, phosphites, phosphonites, benzofuranones, metal stearates, metal oxides, pigments, dyes, organophsophorus compounds, hydroxylamines, flame retardants or mixtures thereof.
